(19) United States
(12) Patent Application Publication
(10) Pub. No.: US 2006/0228433 A1
(43) Pub. Date: Oct. 12, 2006

(54) Method for restoration of gap junctional intercellular connection

(75) Inventors: Yasushi Nakamura, Kyoto (JP); Brad L. Upham, East Lansing, MI (US); James E. Trosko, Okemos, MI (US)

Correspondence Address: Ian C. McLeod;McLeod & Moyne, P.C., 2190 Commons Parkway, Okemos, MI, 48864, US

(73) Assignee: Board of Trustees of Michigan State University, East Lansing, MI (US)

(21) Appl. No.: 11/412,743

(22) Filed: Apr. 27, 2006

Related U.S. Application Data
(63) Continuation-in-part of application No. 10/375,401, filed on Feb. 27, 2003.
(60) Provisional application No. 60/362,562, filed on Mar. 7, 2002.

(51) Int. Cl. A61K 36/68; A61K 31/56

(52) U.S. Cl. 424/738; 514/169

(57) ABSTRACT

A method for restoring gap junctional intercellular communication (GJIC) in the cells of a mammal, including humans. The method includes administering to mammals who have been determined to have a mutation in the ras gene a phytosterol to restore GJIC. The phytosterol can be β-sitosterol, stigmasterol, or mixtures of these phytosterols. Administering the phytosterol compound both restores gap junctional intercellular communication (GJIC) and inhibits anchorage independent growth of mammalian cells which have a mutation in the ras gene.

### CROSS-REFERENCE TO RELATED APPLICATION

This application is a continuation-in-part of U.S. patent application Ser. No. 10/375,401, filed Feb. 27, 2003, and claims benefit of Provisional Application No. 60/362,562 filed Mar. 7, 2002.

### STATEMENT REGARDING FEDERALLY SPONSORED RESEARCH OR DEVELOPMENT

This invention was made in the course of work supported by a National Institute of Environmental Health Sciences Grant No. PA42 ES04911. Therefore, the U.S. Government has certain rights in the invention.

### REFERENCE TO A “COMPUTER LISTING APPENDIX SUBMITTED ON A COMPACT DISC”

Not Applicable.

### BACKGROUND OF THE INVENTION

(1) Field of the Invention

The present invention relates to phytosterols isolated from psyllium which are anti-tumorigenic. The phytosterols restores gap junctional intercellular communication (GJIC) and inhibits anchorage independent growth of mammalian cells which have been transformed with the ras oncogene. The phytosterols are useful as chemotherapy and chemopreventative agents. The phytosterols were identified using a novel method for determining the anti-tumorigenic potential of a compound or composition.

(2) Description of Related Art

Psyllium (Plantago ovata Forsk) or “desert Indian wheat” is a cultivated plant weed, belonging to the Plantaginaceae family, and is native to Iran and India. In the USA, it is distributed in the southwest deserts of Arizona, California, Nevada, Texas and Utah. The psyllium seed husk is primarily used in traditional herbal medicine for colon care because it has high fiber content composed mostly of water-insoluble dietary fiber, hemicelluloses (1). The seed husk also contains water-soluble dietary fibers, which swells in the intestinal tract, and makes a bulky mass absorbing potentially toxic waste and cholesterol in the intestine (1, 2). Currently, psyllium seed husks are mainly used as a dietary supplement to treat hypercholesterolemia, constipation and daily colon care. Many epidemiological studies have been designed to investigate psyllium, which showed cholesterol-lowering effects in persons with hypercholesterolemia. Although the epidemiological evidence is not entirely consistent, dietary fiber has been linked to the prevention of cancers, particularly of the colon and breast (Greenwald et al., Eur. J. Cancer 37: 948-965 (2001); Gerber, J. Natl. Cancer Inst. 88: 857-858 (1996); Terry et al., J. Natl. Cancer Inst. 93: 525-533 (2001)). The underlying mechanisms by which dietary fiber can contribute to cancer prevention are not known. Among some of the potential mechanisms proposed for psyllium are the presence of the anticarcinogenic phytates, isoflavonoids, and protease inhibitors in psyllium fiber, and decreases in the circulation of tumor-promoting estrogens either through suppression of bacterial B-D-glucuronidase activity in the colon and cecum or direct binding of estrogens to fiber (Cohen et al., J. Natl. Cancer Inst. 88: 899-907 (1996)).

Neither the stage nor stages of the multi-step/multi-mechanism nature of the carcinogenic process is known as to where the anticarcinogenic properties of psyllium are effective. Although the effect of psyllium was most pronounced in reducing mammary adenocarcinoma, psyllium also decreased ductal carcinomas (Cohen et al., J. Natl. Cancer Inst. 88: 899-907 (1996)). Since ductal carcinomas are a morphologic continuum from an original initiating event to a fully developed carcinoma (Boone et al., Cancer Res. 52: 1651-1659 (1992)), then the clonal expansion stage of cancer development might be a target of psyllium.

One hypothesis of the tumor promotion mechanism is that the clonal expansion of an initiated cell results from a series of epigenetic events that remove this initiated cell from growth suppression via the inhibition of gap junctional intercellular communication (GJIC) and that activate mitogenic signal transduction pathways (Upham and Wagner, Toxicol. Sci. 64: 1-3 (2001); Trosko and Ruch, Front. Biosci. 3: 208-236 (1998); Rummel et al., Toxicol. Sci. 49: 232-240 (1999)). Gap junctions are channels between contiguous cells allowing the passive transfer of low molecular weight molecules (<1,200), and are made up of protein subunits termed connexins (Goodenough, Annu. Rev. Biochem. 65: 475-502 (1996); Kumar and Gilula, Cell 84: 381-388 (1996)). The species of connexin is selectively expressed in specific organs and cells, and connexin 43 predominantly plays a role of GJIC construction in rat liver epithelial cells.

Connexin genes have been shown to function as tumor suppressor genes (Trosko and Ruch, Front. Biosci. 3: 208-236 (1998); Yamasaki et al., Novartis. Found. Symp. 219: 241-254 (1999)). Transfection of connexin genes into neoplastic cells results in the restoration of GJIC and reversal of the transformed phenotype (de-Feijter-Rupp et al., Carcinog. 19: 747-754 (1998); Huang et al., Cancer Res. 58: 5089-5096 (1998); Hirschi et al., Cell Growth Differ. 7: 861-870 (1996); Rose et al., Carcinog. 14: 1073-1075 (1993); Mesnil et al., Cancer Res. 55: 629-639 (1995); Naus et al., Cancer Res. 52: 4208-4213 (1992)). Similarly, some anticarcinogenic compounds, such as retinoids (Mehta et al., J. Cell Biol. 108: 1053-1065 (1989); Mehta and Loewenstein, J. Cell Biol. 113: 371-379 (1991); Mehta et al., Cell 44: 187-196 (1986); Hossain et al., Carcinog. 10: 1743-1748 (1989)), carotenoids (Zhang et al., Carcinogenesis 12: 2109-2114 (1991)), caffeic acid (Na et al., Cancer Letts. 157: 31-38 (2000)) and lovastatin (Ruch et al., Mol. Carcinog. 7: 50-59 (1993)), are also known to upregulate GJIC, either by preventing the inhibition of GJIC by tumor promoters or by the restoration of GJIC in tumor cells with expressed but non-functional connexins in neoplastic cell lines that result in reversing the transformed phenotype. Green tea extract, which inhibits promotion of tumors in livers (Klaunig, Prev. Med. 21: 510-519 (1992)), also prevents the in vivo inhibition of GJIC in the liver tissues of rats treated with the tumor promoter, pentachlorophenol (Sai et al., Carcinog. 21: 1671-1676 (2000)). Published U.S. Patent Application No. 2001/0024664 A1 to Obukowicz et al. discloses that organic extracts prepared from edible plant materials, including psyllium, contain COX-2 inhibitory compounds which are useful for relieving pain, including pain produced by cancers. There is no suggestion that the organic extracts be used to treat cancers per se.

Notwithstanding the forty-year war on cancer and the deliberate progress which has been made toward improving the prognosis for many types of cancer, cancer remains a killer that continues to terrorize the population. With the recent discoveries of natural plant compounds that have anti-cancer properties, the idea that there might be plant products which will provide even more efficacious anti-cancer compounds has captured the imagination of medical research teams around the world. Therefore, there remains a need for compounds and compositions isolated from natural sources which have anti-cancer and anti-tumor properties.

### SUMMARY OF THE INVENTION

The present invention provides methods of reversing the inhibitory effect of the ras oncogene on gap junctional intercellular communication (GJIC) and the stimulatory effect of the ras oncogene on anchorage independent growth of mammalian cells which have been transformed with the ras oncogene. In other words, the method restores gap junctional intercellular communication (GJIC) and inhibits anchorage independent growth of mammalian cells which have been transformed with the ras oncogene. Possible uses include use as a chemotherapy and chemopreventative agent. The compound was identified using a novel method for determining the anti-tumorigenic potential of a compound or composition.

Therefore, the present invention provides a method for restoring gap junctional intercellular communication (GJIC) in GJIC-deficient tumorigenic cells which comprises: providing a plurality of mammalian cells; determining whether one or more of the plurality of mammalian cells are GJIC-deficient tumorigenic cells that comprise a mutation in the ras gene; and administering to the mammalian cells, if one or more of the mammalian cells have been determined to have the mutation in the ras gene, a phytosterol compound in an amount sufficient to restore the GJIC in the GJIC-deficient tumorigenic cells and thereby inhibit the formation of a tumor. In further embodiments, the phytosterol compound is selected from the group consisting of β-sitosterol, stigmasterol and mixtures thereof. In still further embodiments, the phytosterol compound is from a seed husk powder of Plantago ovata. In still further embodiments, the mammalian cells are human cells. In still further embodiments, the phytosterol compound comprises β-sitosterol administered to the mammalian cells at a concentration of 1 μM to about 10 μM.

The present invention further provides a method for inhibiting tumorigenic cells in a human or lower animal which comprises: providing a phytosterol compound capable of restoring GJIC in GJIC-deficient tumorigenic cells comprising a mutation in a ras gene; and administering the phytosterol compound to the human or lower animal in an amount sufficient to restore the GJIC in the GJIC-deficient tumorigenic cells and thereby inhibit the formation of a tumor. In further embodiment, the phytosterol compound is selected from the group consisting of β-sitosterol, stigmasterol and mixtures thereof. In still further embodiments, the phytosterol compound is from a seed husk powder of Plantago ovata. In still further embodiments, the phytosterol compound is provided in a pharmaceutically acceptable carrier. In still further embodiments, the phytosterol compound comprises β-sitosterol administered in an amount sufficient to achieve a serum concentration of about 1 μM to about 10 μM.

The present invention provides a method for inhibiting tumorigenic cells in a human or lower animal which comprises: providing a phytosterol capable of restoring GJIC in GJIC-deficient tumorigenic cells comprising a mutation in a ras gene; providing a chemotherapeutic agent; and administering the phytosterol and the chemotherapeutic agent to the human or lower animal in an amount sufficient to inhibit the formation of a tumor. In further embodiments, the phytosterol is selected from the group consisting of β-sitosterol, stigmasterol and mixtures thereof. In still further embodiments, the phytosterol is from a seed husk powder of Plantago ovata. In still further embodiments, the phytosterol is provided in a pharmaceutically acceptable carrier. In still further embodiments, the phytosterol compound comprises β-sitosterol administered in an amount sufficient to achieve a serum concentration of about 1 μM to about 10 μM.

The present invention provides a method of chemotherapy in a human or lower animal having a tumor which comprises: providing a phytosterol capable of restoring GJIC in GJIC-deficient tumorigenic cells comprising a mutation in a ras gene; and administering the phytosterol to the human or lower animal in an amount sufficient to inhibit growth of the tumor. In still further embodiments, the phytosterol is selected from the group consisting of β-sitosterol, stigmasterol and mixtures thereof. In still further embodiments, the phytosterol is from a seed husk powder of Plantago ovata. In still further embodiments, the phytosterol is provided in a pharmaceutically acceptable carrier. In still further embodiments, the phytosterol compound comprises β-sitosterol administered in an amount sufficient to achieve a serum concentration of about 1 μM to about 10 μM.

### OBJECTS

It is an object of the present invention to provide methods for restoring gap junctional intercellular communication (GJIC) using phytosterol compounds.

This and other objects of the present invention will become increasingly apparent with reference to the following drawings and preferred embodiments.

### BRIEF DESCRIPTION OF THE DRAWINGS

FIG. 1A is a control which shows that treating WB-Ha-ras cells with ethanol for 48 hours does not restore GJIC. GJIC was measured using the scrape loading dye transfer technique. Bar inset=50 μm.

FIG. 1B shows that treating WB-Ha-ras cells with 1.5 mg/mL of crude psyllium seed husk powder for 48 hours restored GJIC. GJIC was measured using the scrape loading dye transfer technique. Bar inset=50 μm.

FIG. 1C shows that treating WB-Ha-ras cells with 50 μg/mL EtOH extract prepared from crude psyllium seed husk powder for 48 hours restored GJIC. GJIC was measured using the scrape loading dye transfer technique. Bar inset=50 μm.

FIG. 2 shows the dose response of psyllium induced restoration of GJIC in WB-Ha-ras cells. The cells were treated for 48 h with the ethanol extract from the seed husk of psyllium. GJIC was measured using the scrape loading dye transfer technique. Each value represents an average of 3 replicates±standard deviation.

FIG. 3A is a control showing non-GJIC in WB-Ha-ras cells. The cells were treated for 48 hours with ethanol. GJIC was measured using the scrape loading dye transfer technique. Bar inset=50 μm.

FIG. 3B shows the efficacy of psyllium-induced restoration of GJIC (48 hours) in WB-Ha-ras cells treated for 48 hours with the crude powder of the seed husk of psyllium (1.5 mg/mL) from Vitamin world (lot 4920401; Expiration 8/2003). GJIC was measured using the scrape loading dye transfer technique. Bar inset=50 μm.

FIG. 3C shows the efficacy of psyllium-induced restoration of GJIC (48 hours) in WB-Ha-ras cells treated for 48 hours with the crude powder of the seed husk of psyllium (1.5 mg/mL) from GNC (lot 96808; Expiration 9/2005). GJIC was measured using the scrape loading dye transfer technique. Bar inset=50 μm.

FIG. 3D shows the efficacy of psyllium-induced restoration of GJIC (48 hours) in WB-Ha-ras cells treated for 48 hours with the crude powder of the seed husk of psyllium (1.5 mg/mL) from GNC (lot 88815; Expiration 8/2004). GJIC was measured using the scrape loading dye transfer technique. Bar inset=50 μm.

FIG. 4 shows changes in the phosphorylation of connexin 43 in response to different concentrations of the ethanol extract of psyllium seed husk in WB-Ha-ras cells. The cells were treated for 48 hours with the ethanol extract from the seed husk of psyllium. Each lane shows different phosphorylated connexin 43 bands. 15 μg of protein was added to each lane.

FIG. 5A shows the intracellular localization of connexin 43 in WB cells. Bar inset=20 μm.

FIG. 5B shows the intracellular localization of connexin 43 in WB cells without primary antibody. Bar inset=20 μm.

FIG. 5C shows the intracellular localization of connexin 43 in WB cells with Cx43 peptide. Bar inset=20 μm.

FIG. 5D shows the intracellular localization of connexin 43 in WB-Ha-ras cells with Cx43 peptide. Bar inset=20 μm.

FIG. 5E shows the intracellular localization of connexin 43 in WB-Ha-ras cells. Bar inset=20 μm.

FIG. 5F shows the effect of the EtOH extract from psyllium seed husk on the intracellular localization of connexin 43 in WB-Ha-ras cells. The cells were treated for 48 hours with 25 μg/mL of the ethanol extract from the seed husk of psyllium. Bar inset=20 μm.

FIG. 5G shows the effect of the EtOH extract from psyllium seed husk on the intracellular localization of connexin 43 in WB-Ha-ras cells. The cells were treated for 48 hours with 37.5 μg/mL of the ethanol extract from the seed husk of psyllium. Bar inset=20 μm.

FIG. 5H shows the effect of the EtOH extract from psyllium seed husk on the intracellular localization of connexin 43 in WB-Ha-ras cells. The cells were treated for 48 hours with 50 μg/mL of the ethanol extract from the seed husk of psyllium. Bar inset=20 μm.

FIG. 6A shows the GJIC in WB-Ha-ras cells. GJIC was measured using the scrape loading dye transfer technique. Bar inset=50 μm.

FIG. 6B shows effect of the ethanol extract from psyllium seed husk on GJIC in WB-Ha-ras cells. The cells were treated for 48 hours with 50 μg/mL of the ethanol extract from the seed husk of psyllium. GJIC was measured using the scrape loading dye transfer technique. Bar inset=50 μm.

FIG. 6C shows the GJIC in WB cells transformed with neu (WB-neu cells). GJIC was measured using the scrape loading dye transfer technique. Bar inset=50 μm.

FIG. 6D shows effect of the ethanol extract from psyllium seed husk on GJIC in WB-neu cells. The cells were treated for 48 hours with 50 μg/mL of the ethanol extract from the seed husk of psyllium. GJIC was measured using the scrape loading dye transfer technique. Bar inset=50 μm.

FIG. 6E shows the GJIC in WB cells transformed with src (WB-src cells). GJIC was measured using the scrape loading dye transfer technique. Bar inset=50 μm.

FIG. 6F shows effect of the ethanol extract from psyllium seed husk on GJIC in WB-src cells. The cells were treated for 48 hours with 50 μg/mL of the ethanol extract from the seed husk of psyllium. GJIC was measured using the scrape loading dye transfer technique. Bar inset=50 μm.

FIG. 6G shows the GJIC in WB cells transformed with myc-ras (WB-myc-ras cells). GJIC was measured using the scrape loading dye transfer technique. Bar inset=50 μm.

FIG. 6H shows effect of the ethanol extract from psyllium seed husk on GJIC in WB-myc-ras cells. The cells were treated for 48 hours with 50 μg/mL of the ethanol extract from the seed husk of psyllium. GJIC was measured using the scrape loading dye transfer technique. Bar inset=50 μm.

FIG. 7A shows the largest colony formed by WB-H-ras cells in an AIG soft agar assay. One thousand cells were plated onto soft agar and overlaid with 3 mL of medium. After 3 weeks, colonies were stained and the number was counted. Photographs were taken of the largest colony found on each plate. Shown are phase contrast images (40×) of the largest colony formed in soft agar.

FIG. 7B shows the inhibition of anchorage independent of WB-Ha-ras cells growth by 25 μg/mL ethanol extract of psyllium seed husk after 21 days. One thousand cells were plated onto soft agar and overlaid with 3 mL of medium containing the ethanol extract. The medium was renewed with the extract every other day. After 3 weeks, colonies were stained and the number was counted. Photographs were taken of the largest colony found on each plate. Shown are phase contrast images (40×) of the largest colony formed in soft agar in response to 25 μg/ml of the extract.

FIG. 7C shows the inhibition of anchorage independent of WB-Ha-ras cells growth by 50 μg/mL ethanol extract of psyllium seed husk after 21 days. One thousand cells were plated onto soft agar and overlaid with 3 mL of medium containing the ethanol extract. The medium was renewed with the extract every other day. After 3 weeks, colonies were stained and the number was counted. Photographs were taken of the largest colony found on each plate. Shown are phase contrast images (40×) of the largest colony formed in soft agar in response to 50 μg/ml of the extract.

FIG. 7D shows the inhibition of anchorage independent of WB-Ha-ras cells growth by 75 μg/mL ethanol extract of psyllium seed husk after 21 days. One thousand cells were plated onto soft agar and overlaid with 3 mL of medium containing the ethanol extract. The medium was renewed with the extract every other day. After 3 weeks, colonies were stained and the number was counted. Photographs were taken of the largest colony found on each plate. Shown are phase contrast images (40×) of the largest colony formed in soft agar in response to 75 μg/ml of the extract.

FIG. 7E shows the dose response of colony numbers in soft agar in response to psyllium for the assay shown in FIGS. 7A to 7D. Each value represents an average of colony numbers of three replicate plates±standard deviation.

FIG. 8A shows the changes in the ras protein levels after a 48 h treatment with the EtOH extract from psyllium seed husk in WB-Ha-ras cells. The cells were treated for 48 hours with the ethanol extract from the seed husk of psyllium. Each lane was loaded with 15 μg of protein. Shown is a Western blot image of the membrane bound (m-p21ras) and cytosolic (p-p21ras) forms of the ras protein.

FIG. 8B shows a densitometry analysis of the ras protein bands in shown in FIG. 8a.

FIG. 9 shows the effect of the EtOH extract from psyllium seed husk on the intracellular localization of the ras protein in the WB-Ha-ras cells. The cells were treated for 48 hours with the ethanol extract from the seed husk of psyllium. The left panel is the phase contrast images, the middle panel is the fluorescent images of the immunostained ras protein, and the right panel is the merged images from the first two panels. Bar inset=5 μm.

FIG. 10A shows the effect of the ethanol extract from psyllium seed husk on phospho-Erk in WB-Ha-ras cells and the normal WB-cells. The cells were treated for 48 hours with the ethanol extract from the seed husk of psyllium. Each lane was loaded with 15 μg protein. Shown is a Western blot image of Erk.

FIG. 10B shows a densiometry analysis of the Erk bands in FIG. 10A in which phospho-Erk was normalized to total Erk.

FIG. 11 illustrates the chemical structure of β-sitosterol.

FIG. 12 illustrates GJIC activity-guided fractionation of psyllium seed husk. The yield (weight, %) and activity was shown right under the each fraction named A-Q. The spots on TLC were detected visually by a method of sulfuric acid-mist with heat after development with the solvent of n-hexane-ethyl acetate-formic acid (31:9:1)

FIGS. 13 A-D illustrate restoration of GJIC activity of β-sitosterol and stigmasterol in WB-Ha-ras cells treated for 48 hours. The essential GJIC in normal WB cells was shown in FIG. 13A. The WB-Ha-ras cells were treated for 48 h with vehicle (FIG. 13B), 1.0 μg/ml β-sitosterol (FIG. 13C), and 1.5 μg/ml stigmasterol (FIG. 13D). GJIC was measured using the scrape loading dye transfer technique. Bar inset=50 μm.

FIG. 14A and FIG. 14B illustrate the change in the connexin 43 level and its phosphorylation in response to stigmasterol and β-sitosterol. Connexin 43 protein was measured at forty-eight hours (48 h) after addition of stigmasterol and β-sitosterol (1.0 μg/ml=2.4 μM) in WB-Ha-ras. The cells were treated for forty-eight hours (48 h) with. Each lane was loaded with 3.0 μg of protein. FIG. 14A is a Western blot image of the constitutive connexin 43 and phosphorylated form of connexin 43. FIG. 14B is a densitometry analysis of both protein bands in FIG. 14A. Each value represents an average of three replicates±standard deviation (SD).

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The term “gap junctional intercellular communication” or “GJIC” as used herein refers to intercellular communication by means of passive transfer through gap junctions of low molecular weight molecules (molecular weight less than about 1,200) between two or more cells. GJIC can transfer critical regulatory ions and small molecules, such as but not limited to Ca²⁺, c-AMP and glutathione, as well as macro-molecular substrates, such as but not limited to amino acids, sugars and nucleotides.

The term “anchorage independent growth” or “AIG” as used herein refers to the ability of tumor cells to proliferate without firm attachment to a surface. The in vitro assay for AIG described herein is used as an indicator of tumorigenicity.

As used herein, the term “mammal”, “mammals” and “mammalian” refers to both humans and lower mammals. “Lower mammals” are mammals lower than a human (ie. subhuman). For example, the term includes companion animals such as dogs and cats, however the term is not limited to companion animals.

Carcinogenesis has been conceptualized as a multi-step, multi-mechanism process consisting of an initiation, promotion and progression phase. While the exact mechanisms underlying each of these phases are not yet known, as discussed above the reversible inhibition of gap junctional intercellular communication (GJIC) and apoptosis has been hypothesized to be a part of the tumor promotion phase. If this hypothesis is correct, the strategy for efficacious chemoprevention and chemotherapy would be to prevent the down regulation of GJIC by tumor promoting chemicals and to restore GJIC in GJIC-deficient tumor cells (7-9). The present invention provides a method for restoring gap junctional intercellular communication (GJIC) in cells using phytosterol compounds. The phytosterol compounds can comprise β-sitosterol, stigmasterol or mixtures thereof. The compound inhibits unregulated or malignant proliferation of cells which are incompetent for gap junctional intercellular communication (GJIC) in vitro and in vivo and competent for anchorage independent growth (AIG) in vitro and in vivo. For example, cells in which the ras gene has been mutated to be permanently turned on and the cells contain other mutations such that the combination of the ras mutation and the other mutations renders the cells malignant (incompetent for GJIC and competent for AIG). The anti-tumorigenic activity of the compound of the present invention effects (1) a reversal of ras-induced GJIC incompetence (inhibition) and thus, effects a restoration of GJIC between cells with such ras mutations and between cells with such ras mutations and normal cells and (2) a concomitant decrease in ras-induced competence in or stimulation of AIG and thus, effects an inhibition AIG in the cells with such ras mutations.

About 30% of human cancers are associated with mutations in the ras gene which turn the gene permanently on. More specifically, about 90% of pancreatic cancers, about 50% of colon and lung cancers, 50% of thyroid tumors, and about 30% of liver tumors and myeloid leukemias have mutations in the ras gene which turn the gene permanently on. Therefore, abrogating the effect of such ras mutations is an important objective of current chemotherapies for ras-induced cancers and tumors. The most common chemotherapy methods involve the use of cytotoxic chemicals which are used in an amount which will kill rapidly growing cells such as tumor or cancer cells without also killing more slowly growing normal cells. While these cytotoxic chemicals can effectively cause tumor or cancer remissions, their cytotoxic nature usually produce a wide range of undesirable side effects which limits the amount and length of time these chemicals can be used.

The methods of the present invention provides a novel means for treating patients (human or other mammals) with tumors or cancers. Plantago ovata FORSSK. (Plantaginaceae), also known as blond psyllium, indian plantago, ispaghula, psyllium, and spongel seeds, contains a variety of known chemicals some which have known biological activities (Phytochemical Database, U.S. Department of Agriculture, ARS, NGRL, Beltsville Agricultural Research Center, Beltsville, Md.). For example, chemicals which have been identified in psyllium but which have unknown biological activities include 4-O-methyl-glucoronic acid, alsobiuronic acid, D-galacturonic acid, D-xylose, DL-alanine, DL-norleucine, DL-valine, various fats, indicamine, L-arabinose, L-asparagine, L-cysteine, L-lysine, L-rhamnose, linolenic acid, planteose, rhamnose, sterols, and uronic acid. Chemicals that have been identified in psyllium and which have known biological activities include aucubin (antibacterial, antidote (amanitin), antiinflammatory, antioxidant, antiprolactin, antistaphylococcic, candidicide, cathartic, diuretic, hepatoprotective, lactagogue, laxative, paralytic, pesticide, rna-inhibitor, uricosuric); behenic-acid (cosmetic); fructose (antialcoholic, antidiabetic, antihangover, antiketotic, antinauseant, laxative, neoplastic, sweetener); galactose (sweetener); glucose (acetylcholinergic, antiedemic, antihepatotoxic, antiketotic, antivaricose, hyperglycemic, memory-enhancer); lignoceric-acid (antihepatotoxic); linoleic-acid (5-alpha-reductase-inhibitor, antianaphylactic, antiarteriosclerotic, antiarthritic, anticoronary, antieczemic, antifibrinolytic, antigranular, antihistaminic, antiinflammatory, antileukotriene-d4, antimenorrhagic, antims, antiprostatitic, cancer-preventive, carcinogenic, hepatoprotective, hypocholesterolemic, immunomodulator, insectifuge, metastatic, nematicide); mucilage (cancer-preventive, demulcent); myristic-acid (cancer-preventive, cosmetic, hypercholesterolemic gas, lubricant, nematicide); oleic-acid (5-alpha-reductase-inhibitor, allergenic, anemiagenic, antiinflammatory, antileukotriene-d4, cancer-preventive, choleretic, dermatitigenic, flavor fema 1-30, hypocholesterolemic, insectifuge, irritant mll, percutaneostimulant, perfumery); palmitic-acid (5-alpha-reductase-inhibitor, antifibrinolytic, flavor fema 1, hemolytic, hypercholesterolemic, lubricant, nematicide, pesticide, soap); stearic-acid (5-alpha-reductase-inhibitor, cosmetic, flavor fema 2-4,000, hypocholesterolemic, lubricant, perfumery, propecic, suppository); sucrose plant (aggregant, antihiccup, antiophthalmic, antioxidant, atherogenic, collyrium, demulcent, flatugenic, hypercholesterolemic, preservative, sweetener, triglycerigenic, uricogenic, vulnerary); tannins (anthelmintic, antibacterial, anticancer, anticariogenic, antidiarrheic, antidysenteric, antihepatotoxic, antihiv, antihypertensive, antilipolytic, antimutagenic, antinephritic, antiophidic, antioxidant, antiradicular, antirenitic, antitumor, antitumor-promoter, antiulcer, antiviral, cancer-preventive, carcinogenic, chelator, cyclooxygenase-inhibitor, glucosyl-transferase-inhibitor, hepatoprotective, immunosuppressant, lipoxygenase-inhibitor, mao-inhibitor, ornithine-decarboxylase-inhibitor, pesticide, psychotropic, xanthine-oxidase-inhibitor); tyrosine (antidepressant, antiencephalopathic, antiparkinsonian, antiphenylketonuric, antiulcer, cancer-preventive, monoamine-precursor); valine (antiencephalopathic, essential, flavor fema 1,000-2,000); and, xylose (antidiabetic, diagnostic mar, dye).

Because of the ability of the phytosterol to restore GJIC and inhibit AIG in proliferating cells with ras mutations, the compound is useful in chemotherapies and chemopreventative strategies for treating cancers and tumors induced by mutations in ras. Chemotherapeutic uses include not only treatments which rely solely on the effects of the compound but also include treatments where the phytosterol compound is mixed with one or more cytotoxic chemicals useful for chemotherapy treatments. The composition enables the cytotoxic chemicals to be used at concentrations which are less apt to cause unwanted side effects. The composition can also be used with chemotherapy enhancing drugs which are often mixed with chemotherapy chemicals to augment the chemotherapy treatment. Such drugs include statins such as lovastatin, simvastatin, pravastatin, and the like and COX-2 inhibitors such as nimesolide, Iodine, celecoxib, rofecoxib, and the like. Thus, chemotherapeutic compositions comprising the phytosterol compounds include mixtures of the compound with cytotoxic chemicals, mixtures of the compound with cytotoxic chemicals and enhancing drugs, and mixtures of the compound with chemotherapy enhancing drugs.

Chemopreventive uses for the phytosterols β-sitosterol and/or stigmasterol include use as a nutraceutical or dietary supplement for use by persons who may have cells which are predisposed to develop a cancer or tumor which is inducible by one or more mutations in the ras gene. Such persons include those who have cells comprising a mutated ras gene but not mutations in one or more other genes which are associated with cancers or tumors but which would render the cells malignant if mutated or persons who have cells comprising one or more mutations in genes associated with cancers or tumors but do not yet have mutations in the ras gene. The phytosterol compound can also be used by any other person who wishes to reduce the risk of developing a cancer or tumor which is inducible by mutations in the ras gene. In many cases, it is most likely that persons predisposed to develop a ras-inducible cancer or tumor would need to ingest the phytosterol compound on a daily basis. A preferred method of use of a crude composition as a nutraceutical is to test the person or patient for cells which contain a ras mutation and then provide the composition to the person or patient.

The prefered phytosterols for restoring gap junctional intercellular communication (GJIC) in the cells of a mammal (including humans) comprises β-sitosterol, stigmasterol, or mixtures thereof. The phytosterol compounds can be isolated from psyllium seeds to various purity levels. In some embodiments, an extract can be prepared by extracting psyllium seed husk powder with an organic solvent such as ethanol or methanol, preferably ethanol, to produce an organic extract. The organic extract is then filtered to remove fibers and other components not soluble in the organic solvent. A Whatman #1 filter paper or the like is sufficient to filter the organic extract. Next, the organic solvent is removed from the filtrate by evaporation to produce a crude dried composition. The evaporation can be performed under reduced or normal pressure, at room temperature or with mild heating, or combinations thereof. In some embodiments, the crude dried composition can be dissolved or suspended in an organic or aqueous solvent or liquid carrier to provide a solution or suspension which can further include chemotherapeutic chemicals, drugs, nutraceuticals, and mixtures thereof. The crude dried composition can be compounded with a pharmaceutically acceptable carrier. The crude dried composition can be admixed with one or more chemotherapeutic chemicals, drugs, nutraceuticals, and mixtures thereof and the admixture compounded with a pharmaceutically acceptable carrier or dissolved in a solvent. In general, ten grams of psyllium seed husk powder will provide about 100 mg of the dried composition.

For chemotherapeutic use in patients who have a ras-induced cancer or tumor, a composition is provided to the patient in a pharmaceutically acceptable carrier at a dose which is sufficient to restore GJIC and inhibit AIG in the cells comprising the cancer or tumor. While the dose may be dependent on the particular cancer or tumor afflicting the patient, in many applications, the dose provides the composition to the cancer or tumor cells at a concentration of between about 5 μg/mL and 100 μg/mL, preferably between about 25 to 75 μg/mL. Furthermore, the composition can include one or more chemotherapy chemicals and/or enhancing drugs for augmenting chemotherapy treatments such as statins or COX-2 inhibitors. When provided in dried form, the composition or compound is processed with pharmaceutical carrier substances by methods well known in the art such as by means of conventional mixing, granulating, coating, suspending and encapsulating methods, into the customary preparations for oral or rectal administration. Thus, preparations for oral application can be obtained by combining the composition or compound with solid pharmaceutical carriers; optionally granulating the resulting mixture; and processing the mixture or granulate, if desired and/or optionally after the addition of suitable auxiliaries, into the form of tablets or dragee cores.

Suitable pharmaceutical carriers for solid preparations are, in particular, fillers such as sugar, for example, lactose, saccharose, mannitol or sorbitol, cellulose preparations and/or calcium phosphates, for example, tricalcium phosphate or calcium hydrogen phosphate; also binding agents, such as starch paste, with the use, for example, of maize, wheat, rice or potato starch, gelatin, tragacanth, methyl cellulose, hydroxypropylmethyl cellulose, sodium carboxymethyl cellulose and/or polyvinylpyrrolidone, esters of polyacrylates or polymethacrylates with partially free functional groups; and/or, if required, effervescent agents, such as the above-mentioned starches, also carboxymethyl starch, cross-linked polyvinylpyrrolidone, agar, or alginic acid or a salt thereof, such as sodium alginate. Auxiliaries are primarily flow-regulating agents and lubricating agents, for example, silicic acid, talcum, stearic acid or salts thereof, such as magnesium stearate or calcium stearate. Dragee cores are provided with suitable coatings, optionally resistant to gastric juices, whereby there are used, inter alia, concentrated sugar solutions optionally containing gum arabic, talcum, polyvinylpyrrolidone, and/or titanium dioxide, lacquer solutions in aqueous solvents or, for producing coatings resistant to stomach juices, solutions of esters of polyacrylates or polymethacrylates having partially free functional groups, or of suitable cellulose preparations such as acetylcellulose phthalate or hydroxypropylmethylcellulose phthalate, with or without suitable softeners such as phthalic acid ester or triacetin. Dyestuffs or pigments may be added to the tablets or dragee coatings, for example for identification or marking of the various doses of active ingredient.

Anticancer or antitumor preparations comprising the composition or compound which can be administered orally further include hard gelatin capsules, as well as hard or soft closed capsules made from gelatin and, if required, a softener such as glycerin or sorbitol. The hard gelatin capsules can contain the composition or compound in the form of a granulate, for example in admixture with fillers such as maize starch, optionally granulated wheat starch, binders or lubricants such as talcum, magnesium stearate or colloidal silicic acid, and optionally stabilizers. In closed capsules, the composition or compound is in the form of a powder or granulate; or it is preferably present in the form of a suspension in suitable solvent, whereby for stabilizing the suspensions there can be added, for example, glycerin monostearate.

Other anticancer or antitumor preparations to be administered orally are, for example, aqueous solutions or suspensions prepared in the usual manner, which solutions or suspensions contain the composition or compound in the dissolved or suspended form and at a concentration rendering a single dose sufficient. The aqueous solutions or suspensions either contain at most small amounts of stabilizers and/or flavoring substances, for example, sweetening agents such as saccharin-sodium, or as syrups contain a certain amount of sugar and/or sorbitol or similar substances. Also suitable are, for example, concentrates or concentrated suspensions for the preparation of shakes. Such concentrates can also be packed in single-dose amounts.

Suitable anticancer or antitumor preparations for rectal administration are, for example, suppositories consisting of a mixture of the composition or compound with a suppository foundation substance. Such substances are, in particular, natural or synthetic triglyceride mixtures. Also suitable are gelatine rectal capsules consisting of a suspension of the composition or compound in a foundation substance. Suitable foundation substances are, for example, liquid triglycerides, of higher or, in particular, medium saturated fatty acids.

Likewise of particular interest are preparations containing a finely ground composition, preferably having a median particle size of 5 μm or less, in admixture with a starch, especially with maize starch or wheat starch, also, for example, with potato starch or rice starch. They are produced preferably by means of a brief mixing in a high-speed mixer having a propeller-like, sharp-edged stirring device, for example with a mixing time of between 3 and 10 minutes, and in the case of larger amounts of constituents with cooling if necessary. In this mixing process, the particles of the composition are uniformly deposited, with a continuing reduction of the size of some particles, onto the starch particles. The mixtures mentioned can be processed with the customary, for example, the aforementioned, auxiliaries into the form of solid dosage units; i.e., pressed for example into the form of tablets or dragees or filled into capsules. They can however also be used directly, or after the addition of auxiliaries, for example, pharmaceutically acceptable wetting agents and distributing agents, such as esters of polyoxyethylene sorbitans with higher fatty acids or sodium lauryl sulphate, and/or flavoring substances, as concentrates for the preparation of aqueous suspensions, for example, with about 5- to 20-fold amount of water. Instead of combining the composition/starch mixture with a surface-active substance or with other auxiliaries, these substances may also be added to the water used to prepare the suspension. The concentrates for producing suspensions, consisting of the composition/starch mixtures and optionally auxiliaries, can be packed in single-dose amounts, if required in an airtight and moisture-proof manner.

In addition, a composition or compound can be administered to a patient intraperitoneally, intranasally, subcutaneously, or intravenously. In general, for intraperitoneal, intranasal, subcutaneous, or intravenous administration, the composition or compound is provided by dissolving, suspending or emulsifying it in an aqueous or nonaqueous solvent, such as vegetable or other similar oils, synthetic aliphatic acid glycerides, esters of higher aliphatic acids or propylene glycol, alcohols such as ethanol; and if desired, with conventional additives such as solubilizers, isotonic agents, suspending agents, emulsifying agents, stabilizers and preservatives. Preferably, the composition or compound is provided as a component in a composition acceptable for intraperitoneal, subcutaneous, or intravenous use in warm-blooded animals or humans. For example, such compositions can comprise a physiologically acceptable solution such as a buffered phosphate salt solution as a carrier for the composition. Preferably, the solution is at a physiological pH. In particular embodiments, the composition is injected directly into the tumor or perfused through the tumor by intravenous administration.

Anticancer or antitumor preparations comprise a composition or compound at a concentration suitable for administration to warm-blooded animals or humans which concentration is, depending on the mode of administration, between about 0.3% and 95%, preferably between about 2.5% and 90%. In the case of suspensions, the concentration is usually not higher than 30%, preferably about 2.5%; and conversely in the case of tablets, dragees and capsules with the composition or compound, the concentration is preferably not lower than about 0.3%, in order to ensure an easy ingestion of the required doses of the compound.

The treatment of cancers and tumors in patients with the preparations comprising the compound is carried out preferably by one or more administrations of a dose of the compound which over time is sufficient to substantially inhibit the cancer or tumor, that is to say, an amount which is sufficient to cause complete or partial remission of the cancer or tumor. If required, the doses can be administered daily or divided into several partial doses which are administered at intervals of several hours. In particular cases, the preparations can be used prior to, in conjunction with, or following one or more other anticancer or antitumor therapies such as radiation or chemotherapy, or in conjunction with surgical procedures for removing cancers or tumors. The administered dose of the compound is dependent both on the patient (species of warm-blooded animal or human) to be treated, the general condition of the patient to be treated, and on the type of cancer or tumor to be treated.

The compound appears to be preferentially effective in restoring GJIC and inhibiting AIG in cells containing a ras mutation but not cells containing mutations in the neu or src genes, or cells containing a myc-ras mutations. Therefore, prior to treating a patient with a cancer or tumor with the compound, it is prudent to determine whether the cancer or tumor cells is induced by a ras mutation and not by a neu, src, or myc-ras mutations. Immunohistochemical methods are well known for distinguishing the above mutations. Thus, it is preferable to determine whether the cells comprising the cancer or tumor contain a ras mutation.

For chemopreventive use in patients or mammals, including humans, who might be predisposed to developing a ras-induced cancer or tumor and other individuals, the compound is provided either as a component of an aqueous solution or in a pharmaceutically acceptable carrier at a dose which is sufficient to restore GJIC and inhibit AIG in the cells comprising the cancer or tumor. The pharmaceutically acceptable carrier can be any one of the above described carriers. In addition, the compound can be admixed with nutrients which have cancer and tumor inhibiting characteristics such as bee propolis, anthracyanins, lignins, various antioxidants, and the like, or other nutrients which are healthy or necessary for maintaining or establishing health in an individual such as vitamins, enzymes, fats, minerals, and mixtures thereof. The most common means for administering the compound for chemopreventative purposes is orally thus, for most applications, the compound is provided in tablets or capsules such as those described above, in an aqueous solution, or as a powder for mixing with an aqueous solution.

In particular embodiments, the present invention further provides a method for restoring GJIC in cells of a patient or mammal, including humans, which have been identified as having cells which are incompetent in GJIC. The method involves the steps of removing a sample of cells from a patient or mammal, including humans, and testing the cells to determine whether the cells have a mutation in the ras gene. In some embodiments, this can be done immunohistochemically using methods well known in the art and in other embodiments, the DNA from the cells can be isolated and analyzed for mutations in the ras gene by polymerase chain reaction, restriction fragment length polymorphisms, or the like. In further embodiments, the cells can be tested for GJIC incompetence, and AIG competence. For a patient whose cells contain a ras mutation and are GJIC incompetent and AIG competence in vitro. For patients or mammals, including humans determined to contain the ras mutation, the patient or mammal, including humans, is administered a composition comprising an alcohol soluble extract of seed husk powder of psyllium which is free of fiber of the psyllium in an amount sufficient to restore the GJIC. In one embodiment, the composition comprises the alcohol soluble extract of seed husk powder of psyllium which is free of fiber of the psyllium and a pharmaceutically acceptable carrier. Alternatively, the patient or mammal, including humans is administered a composition comprising seed husk powder of psyllium in an amount sufficient to restore the GJIC psyllium seed husk powder to restore GJIC. The above method is useful for treating cancers and tumors in a patient or mammal, including humans, and for inhibiting formation of cancers or tumors in a patient or mammal, including humans.

The present invention further provides an in vitro method for determining whether a compound or composition has the ability to restore GJIC and inhibit AIG in a cell line which is GJIC incompetent and AIG competent. An example of such a cell line is a cell line wherein the cells comprise a ras mutation which renders the cells GJIC incompetent (inhibits GJIC) and renders the cells AIG competent (stimulates AIG). The method entails two separate assays: the first assay measures restoration of GJIC and the second assay measures inhibition of AIG. The above method enabled the discovery of the composition of the present invention. The above method can be used to determine which of the compounds or mixture of compounds which have been identified above might be capable of restoring GJIC and inhibiting AIG.

In the first assay, the cell line is incubated in tissue culture plates in media containing one or more dilutions of the test compound. After about 48 hours, the cells are assayed for restoration of GJIC using the scrape-load dye technique described in Weis et al., Environ. Heath Perspect. 106: 17-22 (1998) and El-Fouly et al., Exp. Cell Res. 168: 422-430 (1987). Briefly, following exposure to the test compound, the cells were washed three times with a buffered solution such as phosphate buffered saline (PBS). A fluorescent dye which cell membrane impermeable is dissolved in the same buffered solution at a concentration of about 1 mg/mL is added to the cells. Three parallel scrapes are then made in the cell monolayer on the plate using a surgical blade to allow passage of the membrane impermeable dye into ruptured cells. After about a three-minute incubation, the cells were washed with buffered solution without the dye to remove extracellular dye and the cells fixed with 4% formalin. Dye migration is visually observed using a fluorescence microscope and compared to controls without the test compound. The distance of dye migration perpendicular to the scrape (that is, between adjacent cells linked only by gap junctions) represents the ability of cells to communicate via GJIC.

In the second assay, about a thousand cells of the cell line in agarose medium are plated onto the top of 0.5% agarose medium in a tissue culture plate. After 1 day, medium containing the test compound is added on top of the agar plates. The medium containing the test compound is renewed every other day. At the end of 3 weeks, colonies are stained overnight with 1 mg/mL of 2-(p-iodophenyl)-3-(nitrophenyl)-5-phenyl-tetrazolium chloride at 37° C. Inhibition of anchorage independent growth is determined by observing a lack of colony growth and/or small size of the colonies compared to controls without the test compound.

The above method requires a cell line which is GJIC deficient and AIG enabled. A preferred cell line has a ras mutation which has rendered the cells GJIC incompetent and AIG competent. For example, the mouse WB-F344 cell line, an immortal cell line which is available from the Health Science Research Resources Bank, Rinku-minamihama 2-11, Sennan-shi, Osaka, Japan under accession number JCRB0193, can be transfected with a recombinant retrovirus vector comprising the v-Ha-ras oncogene and a neomycin-resistant marker as described in de-Feijter et al., Mol. Carcinog. 3: 54-67 (1990) to produce a WB-H-ras cell line which is GJIC deficient and AIG enabled. The WB-H-ras cell line has been available by name from Michigan State University, Department of Pediatrics & Human Development, 243 National Food Safety & Toxicology Center, East Lansing, Mich., since 1990.

A method for identifying compounds which restore GJIC but without consideration of whether the compounds affect AIG (that is, a method which includes the first assay and not the second assay) can use the above WB-H-ras cell line or the HPDE6c7 cell line. The human pancreatic ductal epithelial clone 7 cell line (HPDE6c7) is a clonal population of immortalized HPDE cells derived from HPDE cells which had been immortalized by transfecting the cells with an amphotrophic retrovirus containing human papilloma virus (HPV) 16 genes E6 and E7. The cell line is incompetent for GJIC but shows anchorage dependent growth in vitro. The cell line has been disclosed in U.S. patent application Ser. No. 10/135,801 to Trosko et al., filed Apr. 30, 2002, and deposited under the terms of the Budapest Treaty at the American Type Culture Collection, 10801 University Boulevard, Manassas, Va. as ATCC PTA-4441.

In some embodiments of the invention, the phytosterol compound is used in combination with one or more other anti-inflammatory, anti-viral, anti-fungal, amoebicidal, trichomonocidal, analgesic, anti-neoplastic, anti-hypertensives, anti-microbial and/or steroid drugs or potentiators. Such drugs include triprolidine or its cis-isomer which is used in combination with chemotherapeutic agents; a phytosterol compound and procodazole, 1H-benzimidazole-2-propanoic acid; [β-(2-benzimidazole) propionic acid 2-(2-carboxyethyl)benzimidazole; propazol] which is a non-specific immunoprotective agent active against viral and bacterial infections that is used with the phytosterol compound; or a phytosterol compound and a platinum-containing drug such as cisplatin which binds DNA which interferes with its DNA repair mechanism and thereby causing cellular death. Other drugs which can be used with a phytosterol, and optionally another chemotherapeutic agent, in the methods of the invention include macrophage colony-stimulating factor (M-CSF), 7-thia-8-oxoguanosine, 6-mercaptopurine, vitamin A (retinol), and other known anti-tumor potentiators which can be used in conjunction with the compounds of the above formula include, monensin, an anti-sense inhibitor of the RAD51 gene, bromodeoxyuridine, dipyridamole, indomethacin, a monoclonal antibody, an anti-transferrin receptor immunotoxin, metoclopramide, N-solanesyl-N,N′-bis(3,4-dimethoxybenzyl)ethylenediamine, leucovorin, heparin, N-[4-[(4-fluorphenyl)sulfonly]phenyl]acetamide, heparin sulfate, cimetidine, a radiosensitizer, a chemosensitizer, a hypoxic cell cytotoxic agent, muramyl dipeptide, vitamin A, 2′-deoxycoformycin, a bis-diketopiperazine derivative, and dimethyl sulfoxide other anti-tumor potentiators.

The chemotherapeutic agents which can be used with the phytosterol compound and an optional potentiator are generally grouped as DNA-interactive agents, antimetabolites, tubulin-interactive agents, hormonal agents, and others such as asparaginase or hydroxyarea. Each of the groups of chemotherapeutic agents can be further divided by type of activity or compound. DNA-interactive agents include the alkylating agents, for example, cisplatin, cyclophosphamide, altretamine; the DNA strand-breaking agents, such as bleomycin; the intercalating topoisomerase II inhibitors, for example, dactinomycin and doxorubicin; the nonintercalating topoisomerase II inhibitors, such as etoposide and teniposide; and the DNA minor groove binder plicamycin.

The alkylating agents form covalent chemical adducts with cellular DNA, RNA, and protein molecules and with smaller amino acids, glutathione and similar chemicals. Generally, these alkylating agents react with a nucleophilic atom in a cellular constituent, such as an amino, carboxyl, phosphate, sulfhydryl group in nucleic acids, proteins, amino acids, or glutathione. The mechanism and the role of these alkylating agents in cancer therapy is not well understood. Typical alkylating agents include: nitrogen mustards, such as chlorambucil, cyclophosphamide, isofamide, mechlorethamine, melphalan, uracil mustard; aziridine such as thiotepa; methanesulphonate esters such as busulfan; nitroso ureas, such as carmustine, lomustine, streptozocin; platinum complexes, such as cisplatin, carboplatin; bioreductive alkylator, such as mitomycin, and procarbazine, dacarbazine, and altretamine.

DNA strand breaking agents include Bleomycin. DNA topoisomerase II inhibitors include the following: intercalators, such as amsacrine, dactinomycin, daunorubicin, doxorubicin, idarubicin, and mitoxantrone; and nonintercalators, such as etoposide and teniposide. The DNA minor groove binder is Plicamycin.

The antimetabolites interfere with the production of nucleic acids by one or the other of two major mechanisms. Some of the drugs inhibit production of the deoxyribonucleoside triphosphates that are the immediate precursors for DNA synthesis, thus inhibiting DNA replication. Some of the compounds are sufficiently like purines or pyrimidines to be able to substitute for them in the anabolic nucleotide pathways. These analogs can then be substituted into the DNA and RNA instead of their normal counterparts. The antimetabolites useful herein include: folate antagonists such as methotrexate and trimetrexate; pyrimidine antagonists, such as fluorouracil, fluorodeoxyunridine, CB3717, azacitidine and floxuridine; purine antagonists such as mercaptopurine, 6-thioguanine, pentostatin; sugar modified analogs such as cytarabine and fludarabine; and ribonucleotide reductase inhibitors such as hydroxyurea.

Tubulin interactive agents act by binding to specific sites on tubulin, a protein that polymerizes to form cellular microtubules. Microtubules are critical cell structure units. When the interactive agents bind on the protein, the cell can not form microtubules tubulin interactive agents include colchicine, vincristine and vinblastine, both alkaloids and paclitaxel and cytoxan.

Hormonal agents are also useful in the treatment of cancers and tumors. They are used in hormonally susceptible tumors and are usually derived from natural sources. These include estrogens, conjugated estrogens and ethinyl estradiol and diethylstilbesterol; chlortrianisen and idenestrol; progestins such as hydroxyprogesterone caproate. medroxyprogesterone, and megestrol; and androgens such as testosterone, testosterone propionate, fluoxymesterone, and methyltestosterone.

Adrenal corticosteroids are derived from natural adrenal cortisol or hydrocortisone. They are used because of their anti inflammatory benefits as well as the ability of some to inhibit mitotic divisions and to halt DNA synthesis. These compounds include, prednisone, dexamethasone, methylprednisolone, and prednisolone.

Leutinizing hormone releasing hormone agents or gonadotropin-releasing hormone antagonists are used primarily the treatment of prostate cancer. These include leuprolide acetate and goserelin acetate. They prevent the biosynthesis of steroids in the testes.

Antihormonal antigens include: antiestrogenic agents such as tamoxifen; antiandrogen agents such as flutamide; and antiadrenal agents such as mitotane and aminoglutethimide.

Hydroxyurea, which appears to act primarily through inhibition of the enzyme ribonucleotide reductase, can also be used in combination with the phytosterol compound.

Asparaginase is an enzyme which converts asparagine to nonfunctional aspartic acid and thus blocks protein synthesis in the tumor. Asparaginase can also be used in combination with the phytosterol compound to treat cancer.

Other chemotherapeutic benzimidazoles and griseofulvin can also be used in combination with the phytosterol compound and optionally a potentiator to treat or inhibit the growth of cancer or extend the life span of a animal or human having cancer.

The amount and identity of a chemotherapeutic agent that is used with a phytosterol compound in the methods of the invention will vary according to cellular response, patient response and physiology, type and severity of side effects, the disease being treated, the preferred dosing regimen, patient prognosis, or other such factors.

The phytosterol compound can be used in combination with one or more other agents or combination of agents known to possess anti-leukemia activity including, by way of example, α-interferon; interleukin-2; cytarabine and mitoxantrone; cytarabine and daunorubicin and 6-thioguanine; cyclophosphamide and 2-chloro-2′-deoxyadenosine; VP-16 and cytarabine and idorubicin or mitoxantrone; fludarabine and cytarabine and γ-CSF; chlorambucil; cyclophosphamide and vincristine and (prednisolone or prednisone) and optionally doxorubicin; tyrosine kinase inhibitor; an antibody; glutamine; clofibric acid; all-trans retinoic acid; ginseng diyne analog; KRN₈₆O₂ (anthracycline drug); temozolomide and poly(ADP-ribose) polymerase inhibitors; lysofylline; cytosine arabinoside; chlythorax and elemental enteral diet enriched with medium-chain triglycerides; amifostine; gilvusmycin; or a hot water extract of the bark of Acer nikoense.

The compounds of the above formula can further be administered to an animal or human with one or more imidazolines and optionally one or more of the above drugs or potentiators as a treatment for cellular proliferative diseases. As used herein, antiproliferative agents are compounds, which induce cytostasis or cytotoxicity. Cytostasis is the inhibition of cells from growing while cytotoxicity is defined as the killing of cells. Specific examples of antiproliferative agents include antimetabolites, such as methotrexate, 5-fluorouracil, gemcitabine, cytarabine; anti-tubulin protein agents such as the vinca alkaloids, paclitaxel, colchicine; hormone antagonists, such as tamoxifen, LHRH analogs; and nucleic acid damaging agents such as the alkylating agents melphalan, BCNU, CCNU, thiotepa, intercalating agents such as doxorubicin and metal coordination complexes such as cisplatin and carboplatin.

The following examples are intended to promote a further understanding of the present invention.

### EXAMPLE 1

This example illustrates the discovery of anti-tumorigenic, alcohol-soluble, fiber-free psyllium seed husk compositions of the present invention.

Ethanol extraction of psyllium seed husk was performed as follows. A 10 g sample of psyllium seed husk powder (GNC, Pittsburgh, Pa.) was extracted with 20 mL of pure ethanol at room temperature and then filtered through a Whatman #1 filter. The residue was washed additional two times with 20 mL of ethanol. Ethanol filtrates were combined and evaporated to dryness using a rotary evaporator at 37° C. The yield was 125±25 mg dried weight from 10 g of the material.

Cells were treated with psyllium extract as follows. WB-Ha-ras cells (5×10⁴) were plated in 35-mm culture plates (Corning Inc., Corning, N.Y.) with 2 mL of Dulbecco's modified Eagle's medium (DMEM, Formula No. 78-5470EF, GIBCO Laboratories, Grand Island, N.Y.) containing 5% fetal bovine serum (5% FBS-DMEM) and cultured overnight. Cells were treated with samples of psyllium (as 10 μL of ethanol solution) in 2 mL of 5% FBS-DMEM.

The normal WB-F344 rat liver epithelial cell line was obtained from Drs. J. W. Grisham and M. S. Tsao of the University of North Carolina (Chapel Hill, N.C.). The cell line is an immortalized, diploid non-tumorigenic cell line derived from a male rat that have retained classic liver oval cell markers (Tsao et al., Exp. Cell Res. 154: 38-52 (1984)). The cell line is also available from the Health Science Research Resources Bank, Rinku-minamihama 2-11, Sennan-shi, Osaka, Japan under accession number JCRB0193. The WB-Ha-ras cell line was developed from the transfection of WB-F344 cell line with a retroviral vector containing the v-Ha-ras oncogene and a neomycin-resistant marker as described in de-Feijter et al., Mol. Carcinog. 3: 54-67 (1990). These ras-transformed cells were characterized as GJIC deficient in vitro and as tumorigenic in vivo (de-Feijter et al., Mol. Carcinog. 3: 54-67 (1990)).

The Scrape Load-Dye transfer Assay for determining gap junctional intercellular communication (GJIC) was performed as follows. GJIC was measured using the scrape loading dye transfer technique (Weis et al., Environ. Heath Perspect. 106: 17-22 (1998); El-Fouly et al., Exp. Cell Res. 168: 422-430 (1987)). Briefly, following exposure to psyllium, the cells were washed three times with phosphate buffered saline (PBS). The fluorescent dye, Lucifer yellow (Sigma, St. Louis, Mo.) dissolved in PBS (1 mg/mL) was added to the cells. Three parallel scrapes were made in the cell monolayer using a surgical blade to allow passage of the membrane impermeable dye into ruptured cells. After a three-min incubation, the cells were washed with PBS to remove extracellular dye and were fixed with 4% formalin. Dye migration was observed and digitally photographed at 200× using a Nikon epifluorescence microscope illuminated with an Osram HBO 200W lamp and equipped with a COHU video camera. The program GEL-EXPERT (Nucleotech, San Mateo, Calif.) was used to quantify GJIC by determining the distance of dye migration. The distance of dye migration perpendicular to the scrape (that is, between adjacent cells linked only by gap junctions) represents the ability of cells to communicate via GJIC. GJIC activity was calculated as the fraction of the solvent control, all treatments were tested in triplicate.

Western blots were performed as follows. Proteins were extracted with 20% SDS solution according to the method reported in (Trosko et al., Methods 20: 245-264 (2000)). The protein content was determined with the DC assay kit (Bio-Rad Corp., Richmond, Calif.). The proteins (15 μg) were separated on 12.5% SDS-PAGE (Laemmli, Nature 227: 680-685 (1970)) and electrophoretically transferred from the gel to PVDF membranes (Millipore Corp, Bedford, Mass.) (Upham et al., Carcinog. 18: 37-42 (1997)). Connexin 43, ras and Erk were detected with anti-connexin 43 (Zymed, South San Francisco, Calif.), anti-pan-ras (Ab-2) monoclonal antibody (Oncogene Research Products, Boston, Mass.), and anti-Erk (total and phosphospecific, New England Biolabs, Beverly, Mass.), respectively, using horseradish peroxidase-conjugated secondary antibody (New England Biolabs, Beverly, Mass.), and then observed with super signal west dura extended duration substrate (Pierce, Rockford, Ill.) and ECL detection kit (Amersham, Life Sci, Denver, Colo.).

Immunofluorescence staining of Connexin 43 and Ras 21 was as follows. WB-Ha-ras cells (2×10⁴) were plated in a 4-well glass attached chamber slide (Nalge Nunc International, Naperville, Ill.) with 1 mL of 5% FBS-DMEM and cultured overnight. A 10 μL aliquot of ethanol extract was added to the cell culture medium, and then incubated for an additional forty-eight (48) hours. After the incubation period, cells were washed with phosphate buffered saline (PBS), three (3) times, and then fixed with 3.5% formaldehyde (30 minutes) and washed once with phosphate buffered saline (PBS); permeabilized the membrane with 0.05% saponin/PBS (30 minutes) and then washed once with PBS. After the cells were fixed they were blocked with 10% goat serum (Sigma, St. Louis, Mo.) in PBS for one (1) hour, and then treated with anti-connexin 43 or anti-Ras 21 antibody diluted 1:100 in 1% goat serum in PBS, and incubated on a shaker at 4° C. for 12 hours. The secondary antibody was a Cy3-conjugated rat or mouse antibody IgG (Jackson Immuno Research Laboratories, Inc., West Grove, Pa.), which was diluted 1/200 in 1% goat serum in PBS and incubated in the dark on a shaker at room temperature for 1 hour. The cells were then washed with PBS and mounted with a cover slip using POLY-AQUAMOUNT (Polysciences, Inc., Washington, Pa.). Microscopic images were digitally obtained from an epifluorescence microscope equipped with a CCD camera (Nikon, Tokyo, Japan).

Anchorage independent growth (AIG) assays were performed as follows. A thousand cells in 3.0 mL of 0.33% agarose medium were plated onto the top of 3.0 mL 0.5% agarose medium. After one day, 3 mL of medium containing psyllium was added on top of these agar plates and this medium was renewed every other day. At the end of 3 weeks, colonies were stained overnight with 1 mg/mL of 2-(p-iodophenyl)-3-(nitrophenyl)-5-phenyl-tetrazolium chloride at 37° C.

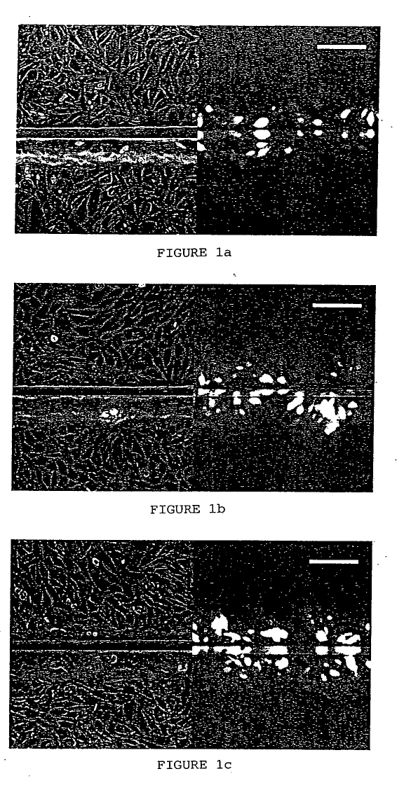

The composition of the present invention restores gap junction intercellular communication (GJIC) in GJIC deficient cells. The WB-Ha-ras cell line has reduced GJIC as compared to the normal WB cell line (de-Feijter et al., Mol. Carcinog. 3: 54-67 (1990)), and the addition of either the crude powder or the ethanol extract greatly increased GJIC to levels comparable to the normal cells as shown by FIGS. 1A to 1C. FIG. 1A shows untreated cells and the absence of GJIC. However, GJIC was restored to the cells when the cells were incubated with crude powder (FIG. 1B) or ethanol extract (FIG. 1C). No difference between the crude and ethanol extract, which was filtered through Whatman #1 paper, was observed suggesting that the result was not purely a fiber effect.

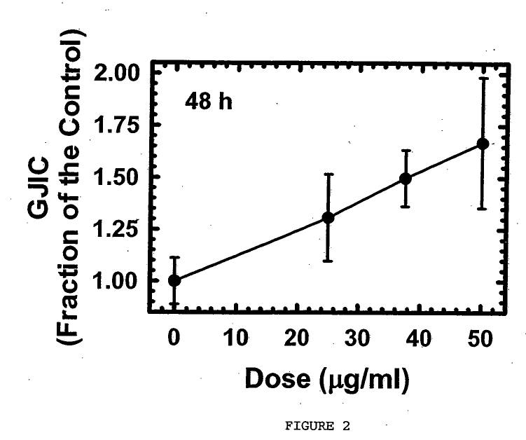

The restoration of GJIC by the ethanol extract of psyllium seed husk was found to be dose dependent (FIG. 2). This dose response was linear in the dose range used (0 to 50 μg/mL).

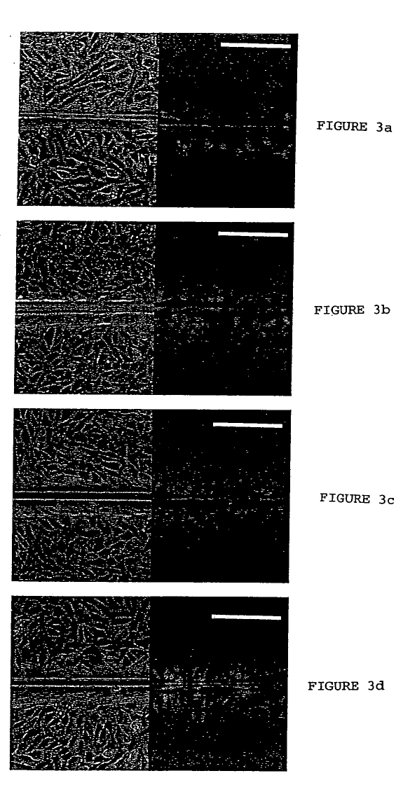

FIGS. 3C to 3D show that the effect of psyllium on GJIC in WB-Ha-ras cells was not specific to the commercial source or lot of the psyllium. For example, restoration of GJIC was observed whether the psyllium was obtained from Vitamin World or GNC (compare FIG. 3B to 3C). However, differences in the magnitude of increasing GJIC were seen between two different lots of psyllium from GNC (compare FIG. 3C to 3D). No cytotoxic effect of psyllium was observed up to 1.5 mg/mL of the crude powder of the seed husk of psyllium, and 50 μg/mL in its ethanol extract in WB-Ha-ras cells.

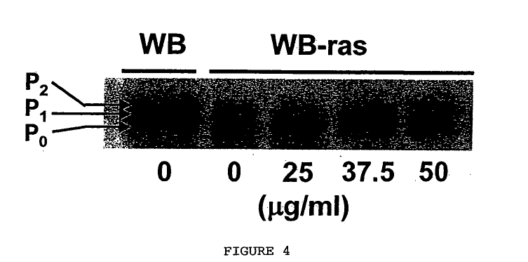

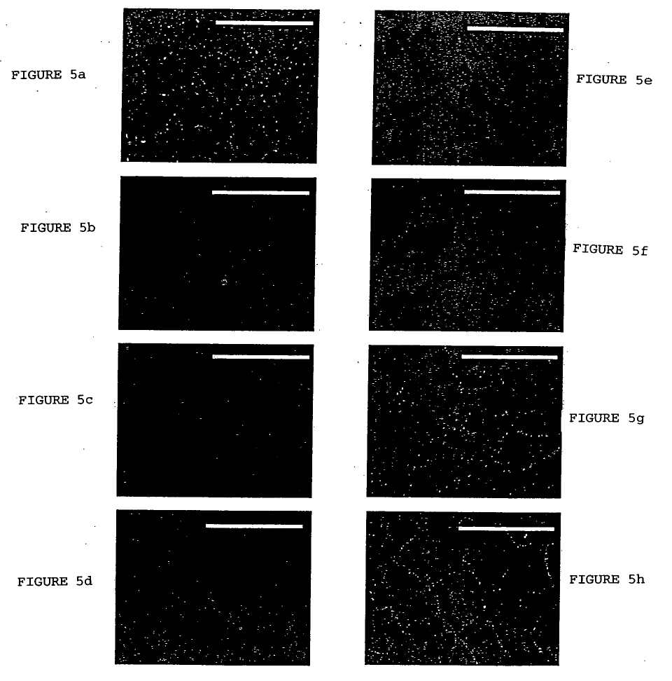

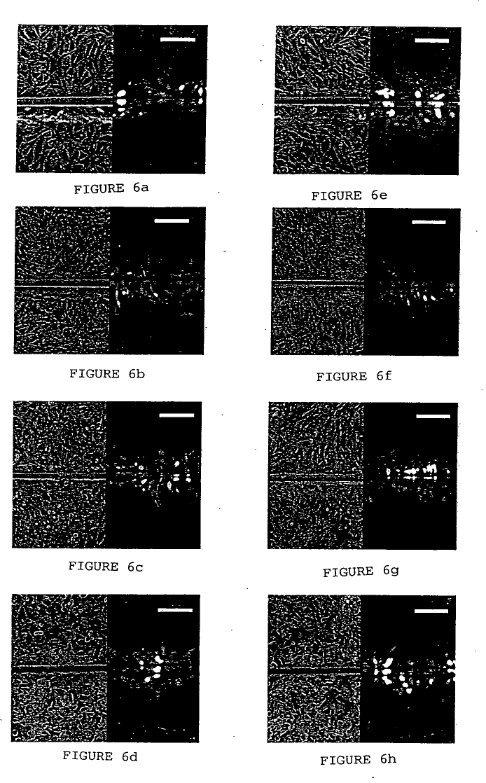

The hypophosphorylated state of the connexins in the WB-Ha-ras cells was reversed back by 50 μg/mL of the ethanol extract of psyllium to levels similar to those of normal WB-cells, which contain both hypophosphorylated and hyperphosphorylated states of the connexins (FIG. 4). Similarly, as shown in FIGS. 5A to 5H, the ethanol extract of psyllium restored the intracellular localization of Cx43 from the cytoplasm of the untreated WB-Ha-ras cells back to the plasma membrane found in normal WB cells. Compare the intracellular location of Cx43 in the untreated WB-Ha-ras cells in FIG. 5E to its location in the cells following treatment with 25 μg/mL of ethanol extract (FIG. 5F), with 37.5 μg/mL (FIG. 5G), and with 50 μg/mL of extract (FIG. 5H) which is similar to its location in normal WB cells (FIG. 5A). The intensity and localization of immunostaining of Cx43 also showed a dose response indicating a normal intracellular pattern at a dose of 50 μg/mL and an almost normal appearance at a dose of 37.5 μg/mL. The effect of psyllium on GJIC was specific to the Ha-ras oncogene (FIG. 6A to 6H). GJIC was not restored by the ethanol extract in WB cells transfected with neu, src, and myc-ras (FIGS. 6D, 6F, and 6H, respectively).

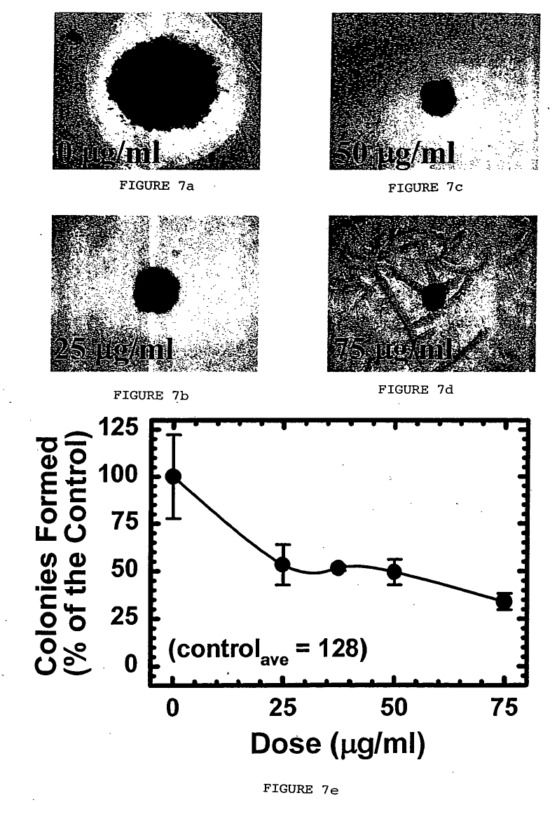

The composition of the present invention inhibits anchorage independent growth (AIG). FIGS. 7A to 7D show that the ethanol extract of psyllium greatly reduced the size of the colonies formed by WB-Ha-ras cells on soft agar and that this effect increased as the dose of extract was increased from 25 μg/mL to 75 μg/mL. The photographic images shown in FIGS. 7A to 7D were representative samples of the plates. Similarly, a dose-dependent response was seen on the number of colonies formed in response to the ethanol extract of psyllium (FIG. 7E). At 75 μg/mL of extract, the number of colonies was about one quarter the number of colonies for the non-treated WB-Ha-ras cells. Normal WB cells do not form colonies on soft agar.

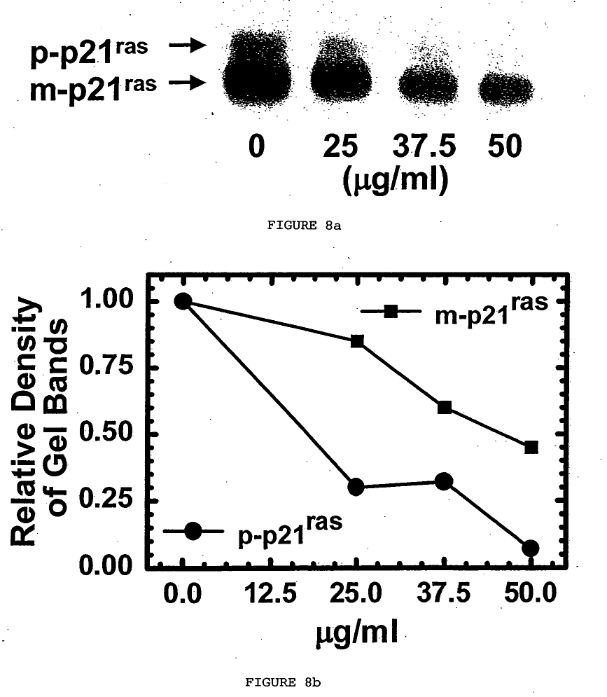

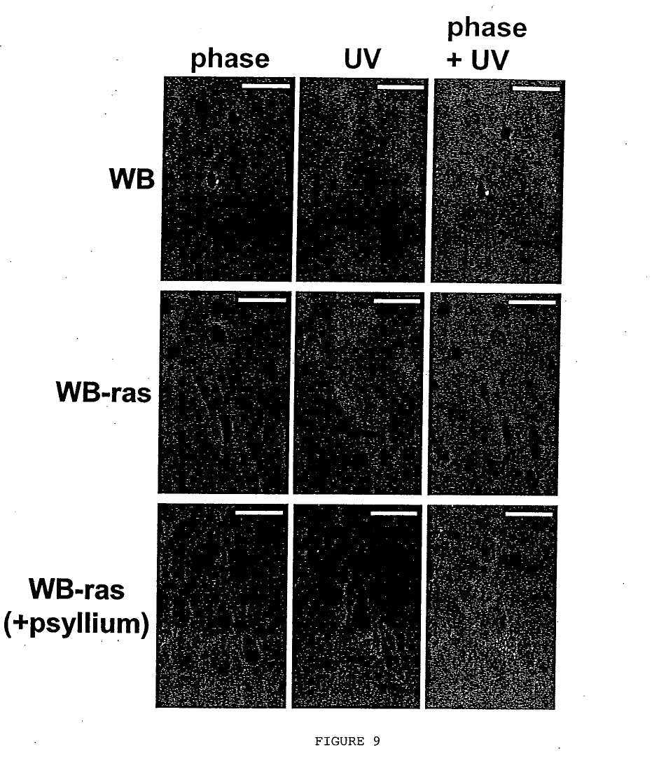

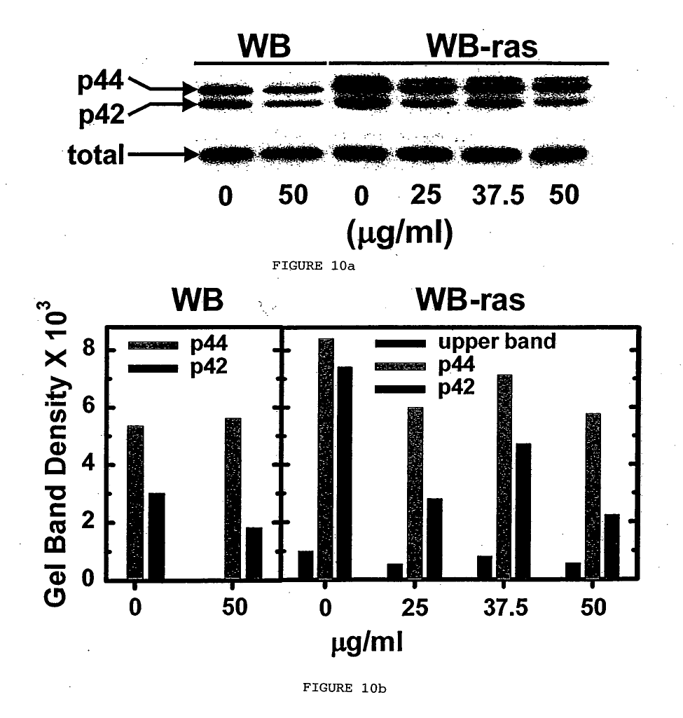

The ethanol extract of the psyllium greatly decreased the level of both the membrane (m-p21ras) and cytosolic (p-p21ras) forms of the ras protein at the noncytotoxic doses ranging between 0-50 μg/mL (FIGS. 8A and 8B). The intracellular-immunohistochemical localization of the ras protein in the normal WB cells was primarily on the plasma membrane in contrast to the more cytosolic localization in the ras transfected cells (FIG. 9). Treatment of WB-Ha-ras cells with the ethanol extract of psyllium resulted in shifting the immunostaining of ras protein from the cytoplasm to the plasma membrane. However, the Western blots (FIG. 8A) indicated that the predominant form of the ras protein in WB-ras was the membrane form of ras (m-p21ras). The ethanol extract of psyllium decreased the level of phosphorylation of Erk1 (p44) and Erk2 (p42) in both the normal WB and the WB-Ha-ras cells (FIGS. 10A and 10B). In addition to the p44 and p42 bands, the WB-Ha-ras also exhibited a small band above p44. The significance of this extra band was not determined, but psyllium had very little effect on this band. Although both p44 and p42 were both affected by psyllium, p42 decreased much more than the p44 band, which was at a higher level (approximately a 1:1 ratio of p44/p42) to begin with in the WB-Ha-ras cells as compared to the normal WB cells that had approximately a 1:0.5 ratio of p44:p42. The psyllium had no affect on the p44 band in the normal WB cells. The densitometry analyses were done on x-ray film exposed to the chemiluminescent bands at several times to assure that the measurement is in the linear range of the film.

The anticarcinogenic mechanism of the ethanol extract of psyllium is not known. Our results suggest that the extract of psyllium could play an important role in preventing full tumorigenic effects of the Ha-ras oncogene. The Ha-ras oncogene is known to bypass the ligand-induced activation of the extracellular receptor kinase (Erk)-mitogen activated protein kinase (MAPK) pathway (McCormick, Trends Cell Biol. 9: M53-M56 (1999)). In addition to the activation of MAPK pathways, cell proliferative events also requires the removal of a cell, such as an initiated cell, from the suppression of growth by neighboring normal cells via the blockage of gap junctional communication (Trosko and Ruch, Front. Biosci. 3: 208-236 (1998); Mehta et al., Cell 44: 187-196 (1986); Goldberg and Bertram, In Vivo. 8: 745-754 (1994)). Numerous studies have shown that transfection of normal cells with oncogenes, including ras, results in a decrease in GJIC, as well as developing tumorigenic phenotypes such as, loss of contact inhibition and AIG, high rates of cell proliferation, and induction of tumors in nude mice (Na et al., Cancer Letts. 157: 31-38 (2000); Trosko et al., Toxicol Lett. 102-103: 71-78 (1998); Jou et al., Carcinog. 16: 311-317 (1995); de-Feijter et al., Mol. Carcinog. 16: 203-212 (1996)). We showed that the extract of psyllium significantly restored GJIC in the Ha-ras transfected F344-WB rat liver epithelial cell line. This restoration of GJIC correlated with a decrease in AIG of these cells in soft agar.

Considerable evidence supports the hypothesis that inhibition of GJIC is fundamental to tumor promotion (Trosko and Ruch, Front. Biosci. 3: 208-236 (1998)), thus, suggesting that the anticarcinogenic properties of the extract of psyllium could be linked, at least in part, to effects on GJIC. A common property of tumor promoters is that they inhibit GJIC, while many anticarcinogenic compounds either block the inhibitory effects of promoters or directly restore GJIC, thereby counteracting the inhibition of GJIC by promoters (Ruch and Trosko, Drug Metab. Rev. 33: 117-121 (2001)). Structural activity relationship models demonstrated a high concordance of carcinogenic activity of compounds with their inhibitory properties of GJIC (Rosenkranz et al., Mutat. Res. 381: 171-188 (1997)). Transfection of oncogenes such as ras, neu and src but not myc into normal cells results in a reduction of GJIC of 50% or more (Jou et al., Carcinog. 16: 311-317 (1995); de-Feijter et al., Mol. Carcinog. 16: 203-212 (1996); El-Fouly et al., Mol. Carcinog. 2: 131-135 (1989); de-Feijter et al., Mol. Carcinog. 5: 205-212 (1992)). Although myc alone does not decrease GJIC, cotransfection of myc with ras results in the complete abolition of GJIC (Hayashi et al., Cancer Lett. 128: 145-154 (1998)). When neoplastic cells come into contact with normal communicating cells, they are growth inhibited and transfection of antisense connexin into the normal cells negates the growth inhibitory effect on the neoplastic cells. A connexin 32 knockout mouse exhibited elevated rates of hepatocytes proliferation, and were more susceptible to spontaneous and initiator-induced hepatic tumor formation. A dominant-negative connexin gene completely abolishes GJIC in neoplastic cells and increases the tumorigenicity of these cells (Krutovskikh et al., Mol. Carcinog. 23: 254-261 (1998)). These published results link GJIC function with cancer.

The mechanism of how the extract of psyllium restores GJIC in Ha-ras-induced inhibition of GJIC has not been determined. Alteration in the phosphorylation patterns of connexins have been proposed as a regulatory mechanism of GJIC, however, there are several examples where altered phosphorylation of connexins did not correlate with inhibition of GJIC (Upham et al., Carcinog. 18: 37-42 (1997); Hossain et al., J. Biol. Chem. 274: 10489-10496 (1999); Hossain et al., J. Cell Physiol. 179: 87-96 (1999)). Furthermore, inhibition of GJIC by environmental contaminants does not always alter the phosphorylation status of connexins (Sai et al., Cancer Lett. 130: 9-17 (1998); Suzuki et al., Nutr. Cancer 36: 122-128 (2000); Upham et al., Int. J. Cancer 78: 491-495 (1998)). The connexins of the WB-Ha-ras cells show a hypophosphorylated protein as well as low molecular weight bands that appear under the Po band. Our results show that the extract of psyllium greatly restored the normal phosphorylation pattern of the Cx43 protein comparable to that of the normal WB cells. In normal WB cell, histochemical analysis of Cx43 results in punctate plaques on the plasma membrane, which is very low in the WB-Ha-ras cells. The extract of psyllium restored the gap junction proteins to the plasma membrane with plaques appearing the same as the normal WB cells.

At present, there is no definitive hypothesis that satisfactorily explains how growth factors and toxicants alter GJIC making it difficult to determine the mechanism of how psyllium is able to reverse the inhibitory effect of the Ha-ras oncogene, particularly since we do not know how the Ha-ras oncogene actually inhibits GJIC. However, growth factor- or ras oncogene-dependent inhibition of GJIC occurs, in part, through the MEK/Erk pathway (Warn et al., J. Biol. Chem. 273: 9188-9196 (1998); Quilliam et al., J. Biol. Chem. 274: 23850-23857 (1999)).

Curiously, our current data shows that there is a strong band that appears above the p44-band of Erk, but the identity of this band was not determined and was not greatly affected by the extract of psyllium, and its affect on the transforming properties of Ha-ras is questionable. However, the extract of psyllium did greatly decrease the p42 and p44 bands in WB-Ha-ras cells, although the p42 band was affected to a much greater extent. In the normal WB cells, only the p42 band was greatly reduced in response to the extract of psyllium. Furthermore, the WB-Ha-ras cells had unusually high levels of p42 relative to the p44 band (approximately 1:1) as compared to the normal WB cells, which had an approximate p44:p42 ratio of 0.5. These results suggest that the restoration of an approximate 2:1 ratio of p44 to p42 by the extract of psyllium might be important in restoring GJIC and inhibiting AIG in these cells.

The Western blot data showed that the total level of the ras protein decreased over 90% in the WB-Ha-ras cells as the dose of the extract of psyllium reached 50 μg/mL. Surprisingly, immunohistochemical staining showed that the intracellular localization of the ras protein was primarily in the cytoplasm in WB-ras cells and with increasing doses of the extract of psyllium, the ras protein migrated to the plasma membrane similar to that of the normal WB cells. However, densitometry analysis of Western blot data of the p21ras protein indicated that there was a higher membrane to cytosolic ratio of p21ras, which is consistent with previously reported results indicating that membrane anchorage, via farnesylation, is important for the oncogenic forms of the ras protein to transform cells (Gibbs et al., Breast Cancer Res. Treat. 38: 75-83 (1996); Agarwal et al., Mol. Carcinog. 17: 13-22 (1996)). Possibly, our antibodies were able to detect the SDS-denatured oncogenic ras protein but were unable to detect, in situ, the membrane bound Ha-ras in the cells and the extract of psyllium displaced this oncogenic form of ras allowing for the expression of the normal ras, in which the antibodies had no trouble detecting the non-denatured form of the normal ras.

The extract of psyllium had no effect on GJIC in WB-cells transfected with other oncogenes, such as neu, src, and myc. Even more interesting is the observation that the extract of psyllium had no effect on ras-myc. WB cells transfected with ras-myc exhibits a greater level of transformation as exhibited by the increase of AIG and tumor formation in nude mice, which correlates with an increased inhibition of GJIC, and represents events that occur in later progressions of a tumor (Hayashi et al., Cancer Lett. 128: 145-154 (1998)). In view of many observations that a mutated or activated ras can be detected in the early stages of carcinogenesis (Reuter et al., Blood 96: 1655-1669 (2000)), these results suggests that the extract of psyllium might be more effective at preventing the earlier stages of tumorigenesis than ameliorating the later stages, thus suggesting a chemopreventive rather than a chemotherapeutic role. This chemopreventative effect would be specific to preventing the growth effects of the mutated ras gene and not other oncogenes.

Anchorage independent growth (AIG) is a common phenotype of transformed cell lines. The underlying mechanisms leading to this phenotype in either oncogene transfected cell lines or cell lines derived from tumorigenic tissue is not completely understood but inhibition of intercellular communication through gap junctions has been determined to be one critical event in this transformation process (Ruch and Trosko, Drug Metab. Rev. 33: 117-121 (2001); Trosko and Ruch, Curr. Drug Targets 3: 465-482 (2002)). Consistent with this GJIC-dependent transformation hypothesis, the restoration of GJIC in the WB-Ha-ras cells by the extract of psyllium strongly correlated with the decrease in AIG activity of this cell line. Further, the extract of psyllium significantly decreased the size of the colonies. These results are similar to the effects of the active anti-cancer ingredient found in honeybee propolis, the phenylethyl ester of caffeic acid (CAPE), which also restored GJIC in the WB-Ha-ras cell line and inhibited AIG (Na et al., Cancer Letts. 157: 31-38 (2000)). CAPE also restored the expression of hyperphosphorylated Cx43 and decreased the protein level of p21ras by Western blot analysis similar to our results.

Another question that arises is whether the soluble fiber, the non-fiber, or both components are responsible for chemoprevention. Our results suggest that the extract of psyllium effect on Ha-ras in our cell line does not involve the fiber component. Which compound or compounds are involved has not been determined but a difference between two different lots from the same company suggests that the concentration of the active ingredient or ingredients can fluctuate. This indicates that identification of the active ingredient or ingredients will be critical in assessing the efficacy of various lots in restoring GJIC in cells with active oncogenic ras.

In summary, our results are consistent with the epidemiological evidence that suggests psyllium has anti-tumorigenic activity. One potential mechanism of the anti-tumorigenic activity of extracts of psyllium is its ability to restore normal GJIC in Ha-ras transformed cells, thus restoring the normal flow of cell signaling molecules between contiguous cells that are important in maintaining the homeostatic set point of growth suppression in a tissue. The implication here is that while the extract of psyllium has potential chemotherapeutic benefit, it might be restricted only to those tumors needing activated Ha-ras. Reversal of the effects of Ha-ras but not myc+Ha-ras suggests that the extract of psyllium might have a more important role in chemoprevention rather than chemotherapy. However, dietary prevention strategies will be very important, considering that advancements in the treatment of colon cancer has not changed the 5 year mortality rate at 50% for almost four decades (Wingo et al., CA-Cancer J. Clin. 45: 8-30 (1995)).

### EXAMPLE 2

Many anticarcinogenic compounds might exert their effect by restoring GJIC, and we set out to determine whether psyllium could exert its anticarcinogenic properties by restoring GJIC in a GJIC-deficient Ha-ras transformed rat liver epithelial WB-F344 cell line (WB-Ha-ras). The GJIC activity in WB-Ha-ras cells was restored by an ethanol extract of psyllium seed husk as shown in Example 1 (24). This example shows that β-Sitosterol from psyllium seed husk (Plantago ovata Forsk) restores gap junctional intercellular communication in Ha-ras transfected rat liver cells. We purified compounds from the husks of psyllium seeds (Plantago ovata Forsk; desert Indian wheat) beginning with an ethanol extraction, followed by HP-20 and silica gel chromatography that restored gap junctional intercellular communication (GJIC) in v-Ha-ras transfected rat liver epithelial cell line. GJIC was assessed by a scrape loading dye transfer assay. The active compound was identified as β-sitosterol based on GC retention times and EI-MS spectrum of authentic β-sitosterol. Authentic β-sitosterol restored GJIC in the tumorigenic WB-Ha-ras GJIC-deficient cells at a dose of 2.4 μM. In addition, a similar phytosterol, stigmasterol, also restored GJIC, albeit at a lower activity. β-sitosterol and stigmasterol increased the level of connexin 43 protein (Cx43) and restored phosphorylation of Cx43 to levels similar to the parental non-transfected cell line. We concluded that the restoration of intercellular communication in the GJIC-deficient, tumorigenic WB-Ha-ras cell line by the ethanol soluble fraction of psyllium seed husks is largely due to the presence of the phytosterol, β-sitosterol. Therefore, dietary modulation of cancer by β-sitosterol is proposed.

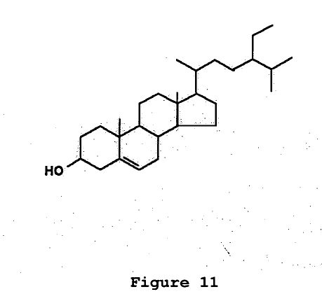

In this example, we purified the ingredient which restored GJIC from the ethanol extract of psyllium via a bioassay-guided fractionation scheme that used a GJIC-deficient WB-Ha-ras cell line to detect GJIC restoring activity of the eluted fractions collected from HP-20 and silica-gel column chromatography, and preparative thin layer chromatography. The compound identified in the purified fraction exhibiting the highest activity in restoring GJIC was a plant sterol, β-sitosterol (chemical structure shown in FIG. 11). Finally, we confirmed that authentic β-sitosterol increased the level of connexin 43 (the GJIC constructed protein) and its active form (phosphorylated connexin 43), which strongly suggests that β-sitosterol is the primary ingredient of psyllium contributing to the restoration of GJIC in the tumorigenic WB-Ha-ras cell line.

Chemicals: β-Sitosterol (99.5% GC pure grade) and stigmasterol (98.8% GC, pure grade) were purchased from Tama Biochemical Co. Ltd. (Tokyo, Japan). Lucifer yellow-CH was purchased from Sigma (St. Louis, Mo.). Psyllium seed husk powder was purchased from Vitamin World Inc. (Ronkonkoma, N.Y.).

Cell Lines and Culture: The WB-F344 rat liver epithelial cell line, obtained from Drs J. W. Grisham and M. S. Tsao of the University of North Carolina (Chapel Hill, N.C.), is a diploid non-tumorigenic cell line derived from a male rat that have retained classic liver oval cell markers (25). The WB-Ha-ras cell line was developed from the transfection of WB-344 cell line with a retroviral vector containing the v-Ha-ras oncogene and a neomycin-resistant marker (26). The cells were characterized as GJIC deficient in vitro and as tumorigenic in vivo.

Treatment of Cells with Sample: WB-Ha-ras cells (5×10⁴) were plated in 35-mm diameter culture plates (Becton Dickinson Labware, Franklin Lakes, N.J.) with 2 ml of modified Eagle's medium (Formula No. 78-5470EF, GIBCO Laboratories, Grand Island, N.Y.) containing 5% fetal bovine serum (5% FBS-DMEM) and cultured overnight, and then treated with samples (as 2.5 μl of ethanol solution) in 2 ml of 5% FBS-DMEM for forty-eight hours (48 h). The reason we chose the forty-eight hour treatment is based on our previous experiments where an ethanol extract of psyllium showed restoration of GJIC at forty-eight hours (24). This is also when the cells are confluent and communicating, albeit at low rates due to the ras oncogene. We always used the sample dose that exhibited no cytotoxicity, and given that the cells are confluent and not proliferating there would be minimal cytostatic effects.

Scrape Load-Dye Transfer Assay: GJIC was measured using the scrape loading dye transfer technique (8). Dye migration was observed and digitally photographed at 200× using a Zeiss Axiovert 25 microscope illuminated with an Osram HBO 50 W lamp and equipped with a Fuji film CCD camera. The distance of dye migration perpendicular to the scrape (i.e. between adjacent cells linked only by gap junctions) represents the ability of cells to communicate via GJIC. GJIC activity was calculated as the fraction of the solvent control. Due to the need to minimize the size of the samples used for the GJIC assay during the purification process and maximize the sample size for the subsequent purification steps, we used only two to three doses, and one to two independent assay of each dose. The active fraction is defined as the fraction at the lowest dose exhibiting a 50% increase in GJIC as compared to the untreated ras cells.

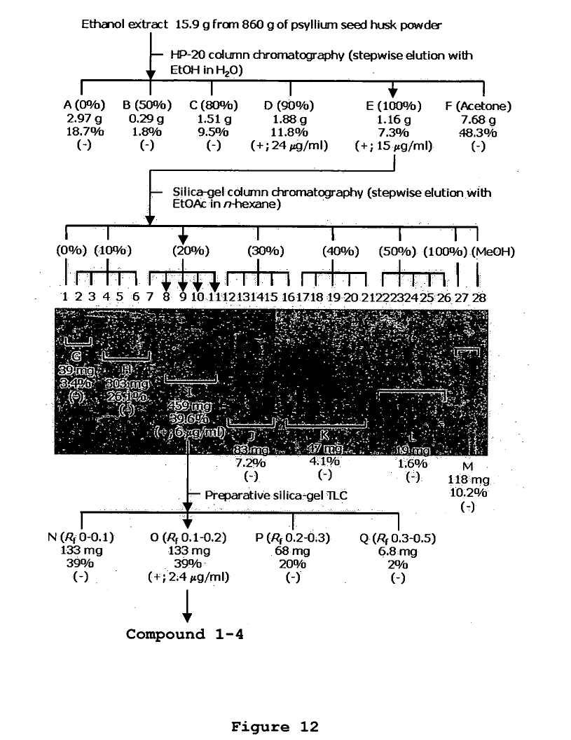

GJIC Assay-Guided Fractionation of Psyllium: Psyllium seed husk powder (860 g) was extracted with ethanol (1.8 L) at room temperature (RT) for twelve hours (12 h), three (3) times. The filtered extract was mixed and evaporated under 40° C. to approximately fifty milliliters (˜50 ml) of an aqueous crude extract solution with a rotary evaporator. The fractionation procedure for each fraction is summarized as illustrated in FIG. 12. The ethanol extract, that showed GJIC restoration was divided into fractions A-F by 160 g of Diaion HP-20 resin column chromatography (Mitsubishi Kasei; φ 3.2×40 cm), using two liters (2 L) of zero, 50, 80, 90, 100% ethanol in water and acetone.

Fraction E (1.16 g), showed an ability to restore GJIC. It was then divided by 32 g of silica gel column chromatography (Merck; Silica gel 60, 35-70 mesh; φ 2.0×30 cm), using 250 ml of each eluent: n-hexane, 10-50% ethyl acetate in n-hexane by 10% stepwise manner, ethyl acetate and methanol. Each eluent of fifty milliliters (50 ml) was collected individually for 10-50% ethyl acetate in n-hexane eluent (# 2-26). Zero percent and 100% ethyl acetate in n-hexane, and methanol eluent were collected and combined individually to 250 ml, and then named # 1, 27, and 28. Each eluent spotted on the TLC plate (Merck; Silica gel 60 F₂₅₄), was developed with the solvent of n-hexane-ethyl acetate-formic acid (31:9:2). Eluents that showed similar spots were combined, from seven fractions (G-M).

Fraction I (459 mg), which showed an ability to restore GJIC, was spotted on preparative-TLC plate (Merck; silica gel 60 F₂₅₄, 20×20 cm), developed with the solvent of n-hexane-ethyl acetate-formic acid (18:2:1). The spot of the compound was detected visually by a method of sulfuric acid-mist with heat, using the edge of the TLC plate. The broad band of Rf value 0-0.1 in fraction N, 0.1-0.2 in fraction 0; 0.2-0.3 in fraction P; and 0.3-0.5 in fraction Q were collected.

Gas Chromatography-Mass Spectrometry (GC-MS): All analyses were performed on a JEOL JMS-AMSUN200 mass spectrometer, coupled on a Hewlett-Packard 6890 gas chromatograph. The capillary column was a DB-5 (25 m×0.2 mm, 0.33 μm film thickness; J&W Scientific, Folsom, Calif., USA). The column oven temperature was held at 60° C. for 5 min and then was increased to 280° C. at 15° C./min.

Protein Extraction and Western Blot Analysis: Proteins of cells were extracted with 20% SDS solution according to the method of Example 1. The protein content was determined with the DC assay kit (Bio-Rad Corp., Richmond, Calif.). The proteins (3 μg) were separated on 7.5% SDS-PAGE (28) and electrophoretically transferred from the gel to PVDF membranes (Millipore Corp, Bedford, Mass.) (29). Connexin 43 was detected with anti-connexin 43 polyclonal antibody (Zymed, South San Francisco, Calif.), using horseradish peroxidase-conjugated secondary antibody (Bio-Rad Corp., Richmond, Calif.), and then observed with the ECL detection kit (Amersham Biosci. Corp., Piscataway N.J.).

Results

Purification of the GJIC Restoration Compound: We purified a compound from psyllium seed husk powder that restored GJIC in a tumorigenic cell line that was deficient in intercellular communication. An overall purification scheme for the compound from the powder of psyllium seed husks that restored GJIC is shown in FIG. 12. The restoration of GJIC activity and the weight of the fractions are shown in Table 1. The restoration of GJIC activity is evaluated by determining the relative distance of dye migration perpendicular to the scrape and was reported as a percent of the solvent control. Based on this criterion, psyllium seed husk powder restored GJIC in WB-Ha-ras cells at 1.5 mg/ml, while the residue didn't show this activity (Table 1). As shown in FIG. 12 and Table 1, the ethanol extract of psyllium powder (15.9 g) showed GJIC restoration activity at 100 μg/ml. In HP-20 column chromatography, Fraction D (80% ethanol elution) and Fraction E (100% ethanol elution) showed the ability to restore GJIC at 24 μg/ml and 15 μg/ml, respectively. Fraction E, which showed stronger activity than Fraction D, was purified further using a mobile phase of 20% ethyl acetate in a silica-gel column. The highest activity (6 μg/ml) that restored GJIC resided in fraction I with a yield of 459 mg. Fraction I was further purified with preparative silica-gel thin layer chromatography (TLC). The highest activity (2.4 μg/ml) for restoring GJIC was in a band (Fraction 0) with an Rf value of 0.1-0.2 and with a yield of 133 mg. In the series of purification steps, the active ingredient in psyllium was successfully concentrated to 625-times from psyllium seed husk powder into Fraction O.

TABLE 1
GJIC Restoration Activities of the Fractions
and Compounds Identified in WB-Ha-ras Cells.

| | Weight | Active dose | Purification |
|---|---|---|---|
| Fraction or compound | (g) | (μg/ml) | fold* |
| Psyllium seed husk powder (start | 860 | 1,500 | 1 |
| material) | | | |
| Ethanol extract | 15.9 | 100 | 15 |
| Fraction E (100% ethanol elution | 1.16 | 15 | 100 |
| in HP-20) | | | |
| Fraction I (20% ethyl acetate | 0.459 | 6 | 250 |
| elution in silica-gel) | | | |
| Fraction O (TLC R_{f}: 0.1-0.2) | 0.133 | 2.4 | 625 |
| Compound 1: Palmitic acid | | Inactive | |
| Compound 2: Linoleic acid | | Inactive | |
| Compound 3: Stearic acid | | Inactive | |
| Compound 4: β-Sitosterol | | 1.0 (2.4 μM) | |

WB-Ha-ras cells (5 × 10⁴) were plated in 35-mm culture plates with 2 ml of 5% FBS-DMEM and cultured overnight. Cells were treated with samples (as 2.5 μl of ethanol solution) in 2 ml of 5% FBS-DMEM for 48 h. GJIC was measured using the scrape loading dye transfer technique.
*Purification fold is expressed in the light of the GJIC restoration activity.

Identification of the GJIC-Restoring Compound in Fraction O: Fraction O contained four compounds (1-4) on the total ion chromatogram from the Low-resolution-gas chromatography-electron ionization (LR-GC-EI) mass spectroscopy analysis. Compound 1, appearing at t_{R} 17.11 min showed the ion peak at an m/z 256 (M)⁺, and prominent fragment ions with masses of 227, 213, 199, 185, 129 and 85. This was identified to be that of palmitic acid by comparing the mass spectrum and retention time in GC-MS with those of an authentic palmitic acid. Authentic palmitic acid did not show activity to restore GJIC at the dose of 10 μg/ml (Table 1). Compound 2, appearing at t_{R} 18.17 min, showed prominent ion peaks at m/z 280 (M)⁺, 264, 220, 207, 193, and 180. This was identified to be that of linoleic acid by comparing the mass spectrum and retention time in GC-MS with those of authentic linoleic acid. Authentic linoleic acid showed no activity of restoring GJIC at the dose of 3 μg/ml (Table 1). Compound 3, appearing at t_{R} 18.37 min, showed prominent ion peaks at m/z 284 (M)⁺, 255, 241, 227, 213, and 129. This was identified to be that of stearic acid by comparing the mass spectrum and retention time in GC-MS with those of authentic stearic acid. Authentic stearic acid did not show any restoration of GJIC activity at the dose of 10 μg/ml (Table 1). Compound 4, appearing at tR 20.35 min showed prominent ion peaks at m/z 414 (M)⁺, 396, 381, 255, 213, 159 and 145. This was identified to be that of β-sitosterol by comparing the mass spectrum and retention time in GC-MS, and the spot color (reddish purple with sulfuric acid-heat) and R_{f}-value in TLC with those of authentic β-sitosterol. The chemical structure of β-sitosterol is shown in FIG. 12. Authentic β-sitosterol showed the ability to restore GJIC at the dose of 1.0 μg/ml (Table 1), which is also close to the specific activity of Fraction O.

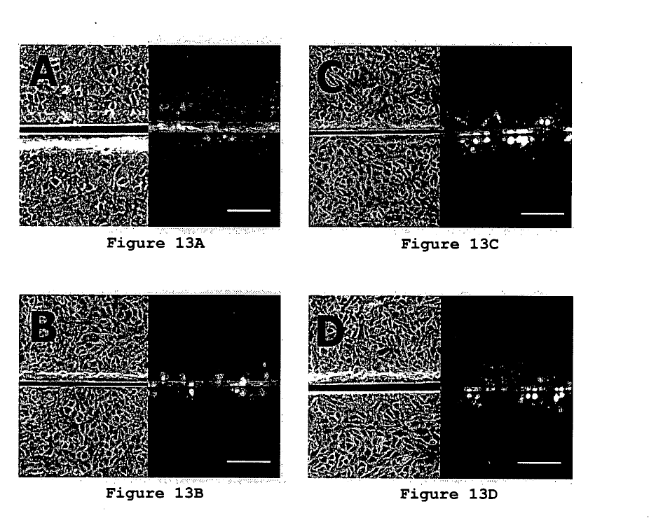

The Activity of β-Sitosterol and Stigmasterol to Restore GJIC: The distance of dye migration perpendicular to the scrape represents the ability of cells to communicate via GJIC (FIG. 13). WB-F344 cells demonstrated excellent GJIC activity (FIG. 13A), as compared to WB-F344 cells transfected with the Ha-ras oncogene (FIG. 13B). We compared the active dose of β-sitosterol with that of stigmasterol (analogue of β-sitosterol). The WB-Ha-ras cells were treated for 48 h with β-sitosterol (C) and stigmasterol (D). Note that 1.0 μg/ml β-sitosterol is equivalent to 2.4 μM and that 1.5 μg/ml stigmasterol is equivalent to 3.6 μM and these doses were both noncytotoxic.

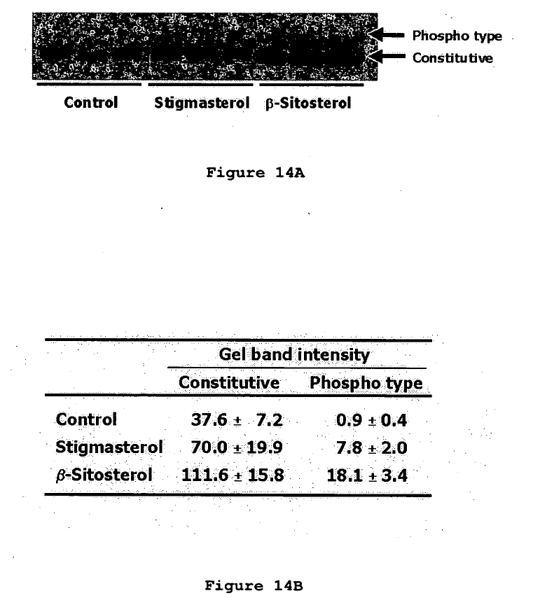

Effect of β-Sitosterol and Stigmasterol on the Amount of the Constitutive and Phosphorylated Connexin 43 Protein in WB-Ha-ras Cells: β-Sitosterol and stigmasterol caused a general increase in the amount of the constitutive connexin 43 protein levels and its phosphorylation form at the dose of 2.4 μM (FIG. 14). The band intensity was 37.6±7.2 in the solvent control, and increased to 70.0±19.9 and 111.6±15.8 in the treatment of WB-Ha-ras cells with stigmasterol and β-sitosterol for 48 h. Similarly, stigmasterol and β-sitosterol increased the band intensity of the phosphorylation form of connexin 43 (active form of connexin 43) from 0.9±0.4 (solvent control) to 7.8±2.0 and 18.1±3.4, respectively. Thus, β-sitosterol induced connexin 43 protein expression more than stigmasterol did at same dose (2.4 μM), and thus, it might be related to the lower dose of β-sitosterol to restore GJIC than stigmasterol.

We discovered that the most active fraction, as determined by the restoration of GJIC in the tumorigenic WB-Ha-ras cells, purified from the seed husks of psyllium contained palmitic, linoleic and stearic acids, and β-sitosterol. This fraction had a purification fold of 625 times that of the crude ethanol extract. Authentic palmitic, linoleic and stearic acids, and β-sitosterol were tested for their ability to restore GJIC in WB-Ha-ras cells resulting in only β-sitosterol showing positive activity at a dose similar to the specific activity of the purified fraction. These results strongly suggest that the active component of ethanol-psyllium extract was β-sitosterol. β-Sitosterol also restored the normal phosphorylation state of the gap junction protein, connexin 43, in the WB-Ha-ras cells as compared to the parental WB-F344 cell line at the same dose needed to restore the activity of GJIC, as well as increasing the overall levels of this protein. In addition, stigmasterol, a structural analogue of β-sitosterol containing a trans A22 unsaturated bond, also restored GJIC and increased connexin 43 levels, albeit at a lower efficiency. These phytosterols may influence connexin 43 levels by either increasing the synthesis of connexin 43 or by decreasing the proteolytic degradation of connexin 43.

Ras proteins (Ha-, K-, and N-) play a significant role on signal transduction involving cell growth, and its mutant form has been found in many tumors. Park et al. reported β-sitosterol (50 μM) decreased cell growth by suppressing the synthesis of DNA that was stimulated in rat fibroblast cells microinjected with Ha-ras, and suggested that β-sitosterol might block the signaling pathway generated by Ha-ras from the cell surface to the nucleus, thus preventing the uncontrolled proliferation of cells that leads to cancer (30). We previously reported that the ethanol extract of psyllium seed husks decreased the level of Ha-ras protein and its farnesylation, followed by suppressed ERK phosphorylation, and thereby inhibited the anchorage-independent colony formation in WB-Ha-ras cells (24). The psyllium effect might be, in part, involved with β-sitosterol, which blocks the signaling pathway mediated by the full tumorigenic effects of the Ha-ras oncogene.

Phytosterols are common components in plant oils, nuts, and cereals (31). The most common components are β-sitosterol, stigmasterol and campesterol, which are structurally similar to cholesterol, but cannot be endogenously synthesized in the human body. Phytosterols in serum are therefore derived from diet exclusively through intestinal absorption (32). While the levels of phytosterols in human serum are reported to typically range from 7-41 μM, these values can be lower due to individual daily diet profiles, thus creating potentially deficient conditions (33). Although we cannot directly extrapolate in vitro doses to in vivo situations, our results Indicate that oncogenic ras mutated cells would probably need to be exposed to 1-10 μM β-sitosterol. Whether these localized concentrations can be achieved with the typical serum levels is unknown, but our results do suggest that daily intake of β-sitosterol-rich plant products could potentially contribute to chemopreventative measures specific to the ras oncogene. Effective in vitro doses similar to ours have also been previously reported. In human colon cancer cells (HCT116), β-sitosterol (10-20 μM) inhibited growth by an induction of apoptosis-mediated proteins, Bax, caspase-3 and caspase-9, and by a decreased expression of the anti-apoptotic Bcl-2 protein (34). In another report, phytosterols or β-sitosterol (16 μM) inhibited growth and induced apoptosis in human prostate or breast cancer cell lines (35, 36).

The psyllium seed husk has been widely used as a supplement to affect colon care, due to its high fiber content including water-insoluble and water-soluble dietary fiber. The present study finds another health-promoting ingredient such as phytosterols (β-sitosterol and stigmasterol), showing an ability to restore GJIC in mutated Ha-ras oncogene transformed liver epithelial cells. Most colon cancer cells have a K-ras mutation (similar function as Ha-ras), and thereby performing uncontrolled signaling pathway in the cells.

The reason psyllium has been traditionally chosen for the herbal care of the colon has been attributed primarily to the high fiber content. However, our findings might either challenge this assumption or might add an additional reason for its presumed therapeutic effects. In addition, Western diet contains only 17-77% phytosterols as compared to the Japanese diet (37-39). In an intervention study with women, it was confirmed that serum phytosterol concentration was increased 20% higher after ingesting plant food-based diet twice a week for 18 weeks (40). It should be noted as a potential conceptual note of caution for dietary supplementation with any bioactive, anti-cancer (or anti-disease) agents that have positive results might only be seen in individuals deficient in a “sufficient”-physiological level of that agent in their body. Supplementing individuals with a bioactive compound that is already at “sufficient” levels might result in no beneficial effects or may even contribute to a negative effect. Lastly, since β-sitosterol identified in this study represents a part of the restoration of GJIC activity found in psyllium, further studies can be done to find other active ingredient(s) should be also identified in another active fraction “Fraction D” (FIG. 12).

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

We claim:

1. A method for restoring gap junctional intercellular communication (GJIC) in GJIC-deficient tumorigenic cells which comprises:
(a) providing a plurality of mammalian cells;
(b) determining whether one or more of the plurality of mammalian cells are GJIC-deficient tumorigenic cells that comprise a mutation in the ras gene; and
(c) administering to the mammalian cells, if one or more of the mammalian cells have been determined to have the mutation in the ras gene, a phytosterol in an amount sufficient to restore the GJIC in the GJIC-deficient tumorigenic cells and thereby inhibit the formation of tumorigenic cells.

2. The method of claim 1, wherein the phytosterol is selected from the group consisting of P-sitosterol, stigmasterol and mixtures thereof.

3. The method of claim 1, wherein the phytosterol is from a seed husk powder of Plantago ovata.

4. The method of claim 2, wherein the mammalian cells are human cells.

5. The method of claim 4, wherein the phytosterol comprises β-sitosterol administered to the mammalian cells at a concentration of 1 μM to about 10 μM.

6. A method for inhibiting formation of tumorigenic cells in a human or lower animal which comprises:
(a) providing a phytosterol capable of restoring GJIC in GJIC-deficient tumorigenic cells comprising a mutation in a ras gene; and
(b) administering the phytosterol to the human or lower animal in an amount sufficient to restore the GJIC in the GJIC-deficient tumorigenic cells and thereby inhibit the formation of tumorigenic cells.

7. The method of claim 6, wherein the phytosterol is selected from the group consisting of β-sitosterol, stigmasterol and mixtures thereof.

8. The method of claim 6, wherein the phytosterol is from a seed husk powder of Plantago ovata.

9. The method of claim 6, wherein the phytosterol is provided in a pharmaceutically acceptable carrier.

10. The method of claim 7, wherein the phytosterol comprises β-sitosterol administered in an amount sufficient to achieve a serum concentration of about 1 μM to about 10 μM.

11. A method for inhibiting tumorigenic cells in a human or lower animal which comprises:
(a) providing a phytosterol capable of restoring GJIC in GJIC-deficient tumorigenic cells comprising a mutation in a ras gene;
(b) providing a chemotherapeutic agent; and
(c) administering the phytosterol and the chemotherapeutic agent to the human or lower animal in an amount sufficient to inhibit the tumorigenic cells.

12. The method of claim 11, wherein the phytosterol is selected from the group consisting of β-sitosterol, stigmasterol and mixtures thereof.

13. The method of claim 11, wherein the phytosterol is from a seed husk powder of Plantago ovata.

14. The method of claim 11, wherein the phytosterol is provided in a pharmaceutically acceptable carrier.

15. The method of claim 12, wherein the phytosterol compound comprises β-sitosterol administered in an amount sufficient to achieve a serum concentration of about 1 μM to about 10 μM.

16. A method of chemotherapy in a human or lower animal having a tumor which comprises:
(a) providing a phytosterol capable of restoring GJIC in GJIC-deficient tumorigenic cells comprising a mutation in a ras gene; and
(b) administering the phytosterol to the human or lower animal in an amount sufficient to inhibit growth of the tumor.

17. The method of claim 16, wherein the phytosterol is selected from the group consisting of β-sitosterol, stigmasterol and mixtures thereof.

18. The method of claim 16, wherein the phytosterol is from a seed husk powder of Plantago ovata.

19. The method of claim 16, wherein the phytosterol is provided in a pharmaceutically acceptable carrier.

20. The method of claim 17, wherein the phytosterol compound comprises β-sitosterol administered in an amount sufficient to achieve a serum concentration of about 1 μM to about 10 μM.
